(19) United States
(12) Patent Application Publication
(10) Pub. No.: US 2023/0273220 A1
(43) Pub. Date: Aug. 31, 2023

(54) METHODS FOR PREDICTION, DETECTION AND MONITORING OF SUBSTANCEUSE DISORDERS AND/OR AN INFECTION

(71) Applicant: NATIONAL HEALTH RESEARCH INSTITUTES, Miaoli County (TW)

(72) Inventors: Yu-Li LIU, Miaoli County (TW); Yen-Feng LIN, Miaoli County (TW); Hsiao-Hui TSOU, Miaoli County (TW); Ren-Hua CHUNG, Miaoli County (TW)

(21) Appl. No.: 17/996,742

(22) PCT Filed: Apr. 20, 2021
(86) PCT No.: PCT/US21/28208

§ 371 Date: Oct. 20, 2022

Related U.S. Application Data
(60) Provisional application No. 63/012,516, filed on Apr. 20, 2020.

(51) Int. Cl. G01N 33/68; G01N 33/94; G01N 33/98; G01N 33/576

(52) U.S. Cl. CPC G01N 33/6863; G01N 33/94; G01N 33/98; G01N 33/576; G01N 2800/52

(57) ABSTRACT

The disclosure provides methods of using biomarkers to predict risk or likelihood and/or prognosis of and/or detect or diagnose substance use disorders or infections and/or monitor progress of substance use disorders and infections.

This application claims priority to International Patent Application No. PCT/US21/28208, filed on Apr. 20, 2021, which claims benefit to and priority of U.S. Provisional Patent Application No. 63/012,516, filed Apr. 20, 2020, and entitled, “A METHOD FOR DIAGNOSING MULTIPLE INDICATIONS IN A SUBSTANCE USE SUBJECT,” the contents of which are referenced in their entirety.

### FIELD OF THE INVENTION

The disclosure relates to biomarkers for predicting the risk or likelihood of an infection and/or a substance use disorder and/or for monitoring progress and/or prognosis of a substance use disorder and/or an infection. Particularly, the disclosure provides methods for predicting the risk or likelihood and/or prognosis of a substance use disorder and/or detecting or diagnosing substance use disorders or infections and/or monitoring progress of a substance use disorder and an infection.

### BACKGROUND OF THE INVENTION

Substance-use or drug addiction disorders produce addiction and brain damage for which there is no reliable and successful therapy or diagnosis. Substance-use disorder is a complex disease that affects a person's brain and behavior and has been diagnosed as a brain dysfunction disease. Dependence upon drugs (addiction) causes major health problems worldwide. For example, alcohol abuse and alcohol dependence can cause liver, pancreatic and kidney disease, heart disease, increased incidence of many types of cancer, insomnia, depression, anxiety, and even suicide. Opioids are a class of drugs that include the illegal drug heroin, synthetic opioids such as fentanyl, and pain relievers available legally by prescription, such as oxycodone, hydrocodone, codeine, morphine, and many others. Opioid use disorder is a chronic lifelong disorder, with serious potential consequences including disability, relapses, and death.

Ketamine, an anesthetic which has provided much needed relief of pain in medical surroundings, has been subject to abuse by individuals leading to their dependence of this drug. Abuse of ketamine can result in a number of systemic manifestations including gastrointestinal issues, depression, and respiratory problems and amnesia. Serious debilitating urinary tract symptoms are also seen frequently in those individuals who abuse ketamine.

US 20200411191 provides technologies for predicting an individual's likelihood of addiction or relapse to pharmaceuticals that include controlled or addictive substances. US 20080171779 relates to the use of 5-HT6 antagonists or pharmaceutical compositions comprising these compounds for preventing relapse into addiction.

However, no effective means for detecting, diagnosing or predicting addiction have been provided.

### SUMMARY OF THE INVENTION

The present disclosure discloses one or more biomarkers or a combination of biomarkers for early detection or diagnosis, and/or progress or prognosis monitoring of substance use disorders and infections. Particularly, the present disclosure surprisingly found that in a biological sample, the level of NFL alone or in combination with one or more CCL11, Nectin-4, MPO and/or ADAM10 can be used as indicators of various symptoms for evaluating substance use disorders.

In one aspect, the present disclosure provides a method for detecting or predicting a substance use disorder and/or monitoring theprogress of the substance use disorder, and/or predicting treatment response or prognosis of substance use disorder in a subject, comprising obtaining a biological sample, and detecting the level of a biomarker NFL in the biological sample, wherein the level of NFL at least 1.0 times higher than a level of a control is indicative of neurotoxicity severity following substance use, and/or predicting treatment responses or prognosis of substance use disorder in a subject.

In one embodiment, a substance use disorder is indicated when the level of NFL in a biological sample is at least 1.0, 1.1, 1.2, 1.3, 1.4, 1.5, 1.6, 1.7, 1.8, 1.9, 2.0 or 2.1 times higher than that in a control. In a further embodiment, the substance use disorder is indicated when the level of NFL in a biological sample ranges from about 1.0 to about 10 times, about 1.2 to about 10 times, about 1.0 to about 80 times, about 1.0 to about 60 times, about 1.5 to about 10 times, 1.5 to about 80 times, 1.5 to about 6 times or 1.5 to about 5 times.

In one embodiment, the plasma or serum level of NFL is higher than 5 pg/ml. In a further embodiment, the plasma or serum level of NFL is higher than 7 pg/ml. In some embodiments, the plasma or serum level of NFL ranges from about 5 pg/ml to about 150 pg/ml, about 5 pg/ml to about 120 pg/ml, about 5 pg/ml to about 100 pg/ml or about 5 pg/ml to about 90 pg/ml. In further embodiments, the substance is alcohol or ketamine.

Certain embodiments of the biomarker include one or more additional biomarkers selected from CCL11 for chronic stress following substance use, Nectin-4 for skin irritation following substance use, MPO for alcohol dependence following substance use and ADAM10 for synaptopathy following substance use. In some embodiments, multiple biomarkers can be used to detect the substance use disorder; for example, the biomarkers comprise NFL and CCL11; NFL, CCL11 and Nectin-4; NFL, CCL11, Nectin-4 and MPO; or NFL, CCL11, Nectin-4, MPO and ADAM10. In further embodiments, in a subject with a substance use disorder, chronic stress is indicated when the level of CCL11 in a biological sample is at least 1, 1.2, 1.3, 1.4, 1.5, 1.6, 1.7, 1.8, 1.9, 2.0, 2.2, 2.4, 2.5, 2.8, or 3.0 times higher than that in a control. In a subject with a substance use disorder, skin irritation is indicated when the level of Nectin-4 in a biological sample is at least 1, 1.2, 1.3, 1.4, 1.5, 1.6, 1.7, 1.8, 1.9, 2.0, 2.2, 2.4, 2.5, 2.8, or 3.0 times higher than that in a control. In a subject with a substance use disorder, alcohol dependence is indicated when the level of MPO in a biological sample is at least 1, 1.2, 1.3, 1.4, 1.5, 1.6, 1.7, 1.8, 1.9, 2.0, 2.2, 2.4, 2.5, 2.8, or 3.0 times higher than that in a control. In a subject with a substance use disorder, synaptopathy is indicated when the level of ADAM10 in a biological sample is at least 1, 1.2, 1.3, 1.4, 1.5, 1.6, 1.7, 1.8, 1.9, 2.0, 2.2, 2.4, 2.5, 2.8, or 3.0 times higher than that in a control.

In a further embodiment, the plasma or serum level of CCL11 is higher than 40 pg/ml. In a further embodiment, the blood level of CCL11 is higher than 50 pg/ml, preferably 60 pg/ml, more preferably, 65 pg/ml. In some embodiments, the blood level of CCL11 ranges from about 40 pg/ml to about 100 pg/ml, about 50 pg/ml to about 100 pg/ml, about 60 pg/ml to about 100 pg/ml or about 65 pg/ml to about 100 pg/ml. In further embodiments, the substance is an opioid or ketamine.

In a further embodiment, the plasma or serum level of nectin-4 is higher than 50 pg/ml. In a further embodiment, the plasma level of nectin-4 is higher than 60 pg/ml, preferably 70 pg/ml, more preferably, 80 pg/ml, 90 pg/ml, 100 pg/ml, 120 pg/ml, 130 pg/ml, 140 pg/ml or 150 pg/ml. In some embodiments, the plasma level of nectin-4 ranges from about 50 pg/ml to about 250 pg/ml, about 60 pg/ml to about 250 pg/ml, about 70 pg/ml to about 250 pg/ml or about 70 pg/ml to about 150 pg/ml. In further embodiments, the substance is an opioid or ketamine.

In a further embodiment, the plasma or serum level of MPO is higher than 30 pg/ml. In a further embodiment, the plasma or serum level of MPO is higher than 40 pg/ml, preferably 50 pg/ml. In some embodiments, the plasma or serum level of MPO ranges from about 30 pg/ml to about 200 pg/ml, about 40 pg/ml to about 200 pg/ml, about 50 pg/ml to about 250 pg/ml, about 30 pg/ml to about 150 pg/ml, about 40 pg/ml to about 150 pg/ml or about 50 pg/ml to about 150 pg/ml. In further embodiments, the substance is an opioid or alcohol.

In a further embodiment, the plasma or serum level of ADAM10 is higher than 15 ng/ml. In a further embodiment, the plasma level of ADAM10 is higher than 20, 22, 24, or 25 ng/ml. In some embodiments, the plasma level of ADAM10 ranges from about 15 ng/ml to about 200 ng/ml, about 18 ng/ml to about 200 ng/ml, about 20 ng/ml to about 150 ng/ml, about 20 ng/ml to about 140 ng/ml, about 20 ng/ml to about 120 ng/ml or about 20 pg/ml to about 100 pg/ml. In further embodiments, the substance is an opioid or alcohol.

Certain embodiments of the substance use disorder include, but are not limited to, drug addiction, drug abuse, drug habituation, drug dependence, withdrawal syndrome, chronic substance use and overdose.

Certain embodiments of the substance include, but are not limited to, alcohol, ketamine, opiate, opioid, cocaine, morphine, amphetamines, nicotine, cotinine, heroin, amphetamine, methamphetamine, cannabis, cannabinoid, narcotic analgesic combinations, drug overdose, or chronic substance use.

In one embodiment, the level of the biomarker is detected by incubating a protein or a peptide of the biomarker in the sample with an antibody specifically binding the protein or the peptide and measuring the expression level of the protein or the peptide. In a further embodiment, the level is measured by immunofluorescent assay, enzyme immunoassay or radioimmunoassay.

In some embodiments, the biological sample is plasma, a tissue, cell, blood, urine or serum. In a further embodiment, the biological sample is plasma or serum.

In one embodiment, the subject has neurotoxicity. In another embodiment, the subject suffers from an infection caused by the hepatitis virus or human immunodeficiency virus.

In another aspect, the present disclosure provides a method for detecting or predicting an infection caused by the hepatitis virus or human immunodeficiency virus and/or monitoring the progress of the infection, and/or predicting treatment response or prognosis of the infection in a subject, comprising obtaining a biological sample, and detecting the level of a biomarker IP-10 and optional CDH2, IL-7 and/or Caspase-10 or any combination thereof in the biological sample, wherein the level of the biomarker higher than a level of a control is indicative of the infection, or treatment response, progress or prognosis of the infection.

In one embodiment, the hepatitis virus is hepatitis B virus (HBV) or hepatitis C virus (HCV).

In some embodiments, the biomarker comprises IP-10 and CDH2; IP-10, CDH2 and IL-7; or IP-10, CDH2, IL-7 and Caspase-10; IP-10, CDH2 and IL-7; IP-10, CDH2 and Caspase-10; or IP-10, IL-7 and Caspase-10.

In one embodiment, the level of the biomarker is detected by incubating a protein or a peptide of the biomarker in the sample with an antibody specifically binding the protein or the peptide and measuring the level of the protein or the peptide. In a further embodiment, the level is measured by immunofluorescent assay, enzyme immunoassay or radioimmunoassay.

In some embodiments, the biological sample is plasma, a tissue, cell, blood, urine or serum. In a further embodiment, the biological sample is plasma or serum.

In a further embodiment, the level of IP-10 is higher than 510 pg/ml. In a further embodiment, the level of IP-10 is higher than 550 pg/ml, 600 pg/ml, 650 pg/ml, 700 pg/ml, 750 pg/ml, 800 pg/ml, 850 pg/ml, 900 pg/ml, 950 pg/ml, 1,000 pg/ml, 1,050 pg/ml, or 1,100 pg/ml.

In a further embodiment, the level of CDH2 is higher than 10 pg/ml. In a further embodiment, the level of CDH2 is higher than 12 pg/ml or 14 pg/ml.

In a further embodiment, the level of IL-7 is higher than 4 pg/ml. In a further embodiment, the expression level of IL-7 is higher than 5 pg/ml.

In another aspect, the present disclosure also provides a kit for detecting or predicting a substance use disorder and/or monitoring theprogress of the substance use disorder, and/or predicting treatment response or prognosis of substance use disorder as described herein. In a further embodiment, the kit can be used to detect or predict an infectious disease following substance use disorder.

In a further aspect, the present disclosure also provides a kit for detecting or predicting an infection caused by the hepatitis virus or human immunodeficiency virus and/or or monitoring the progress of the infection, and/or predicting treatment response or prognosis of the infection as described herein.

The kit described herein can further comprise an antibody specifically binding the protein or the peptide and a second antibody specifically binding the protein or the peptide. Preferably, the antibody is tagged. In a further embodiment, the tag is a radioactive atom, a fluorescent molecule, an enzyme, or an insoluble solid phase.

### BRIEF DESCRIPTION OF THE DRAWINGS

FIG. 1A-1B are curve graphs showing receiver operative characteristic (ROC) curves of IP-10 in addicted subjects with HCV or HIV infection. (A) ROC curve for 309 addicted subjects with HCV infection and 18 addicted subjects without HCV infection. (B) ROC curve for 74 addicted subjects with HIV infection and 255 addicted subjects without HIV infection in methadone maintenance treatment patients. (AUC, area under the curve; CI, confidence interval).

FIG. 2 is a curve graph showing ROC curve analyses of plasma levels of caspase-10 with HBV infection.

FIG. 3 shows ROC curve analyses of plasma levels of ADAM10 with MMT patients and controls.

FIG. 4A-4C are graphs showing the correlation between plasma level of nectin-4 and skin irritation indication in addicted subjects. (A) The plasma nectin-4 level among age- and gender-matched normal subjects (Ctr), medication-free abstinent former heroin users (Abstinent), MMT subjects with self-report of skin irritation (Skin irritation) and without skin irritation (Non-skin irritation). (B) A suggested cut-off level in correlation with plasma level of nectin-4 is shown using ROC curve analyses. (AUC, area under the curve; CI, confidence interval) (C) The correlation between plasma nectin-4 levels and in age- and gender-matched.

FIG. 5 is a curve graph showing ROC curve analyses of plasma levels of CCL2, CCL11, CCL22, and FGF-2 with a cut-off at age 45.

FIG. 6A-6C are scatter plots demonstrating the correlations between age and plasma CCL11 concentration in addicted subjects undergoing methadone maintenance treatment (MMT) and normal subjects. (A) All of the MMT addicted subjects versus normal subjects. (B) Positive addicted subjects subgrouped by the urine morphine test versus normal subjects. (C) Negative addicted subjects subgrouped by the urine morphine test versus normal subjects.

FIG. 7 is a curve graph showing ROC curve analyses of plasma levels of NFL with ketamine patients and controls.

FIGS. 8A and 8B show the comparison of NFL levels between healthy controls, patients with ketamine dependence without a lifetime history of MDD (without MDD), and those with a history of MDD (with MDD) by Mann-Whitney U test without adjustment (A), and ANCOVA with Tukey-Kramer multiple comparison test after adjustment for BMI and smoking status (B). (Abbreviations: N: number of patients; SD: Standard deviation).

FIG. 9 shows the differences of nectin-4 levels between patients with KD with and without LUTS and normal controls.

FIG. 10 shows the ROC curve analyses of serum levels of nectin-4 with ketamine patients and controls.

FIGS. 11A to 11C show the ROC curve analyses of plasma levels of CCL11, NFL and MPO with alcohol dependence patients and controls.

### DETAILED DESCRIPTION OF THE INVENTION

Before the present invention is described, it is to be understood that this invention is not limited to the particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 5%, preferably 3%, more preferably 1%.

As used in this specification and the appended claims, the singular forms “a,” “an” and “the” include plural references unless the content clearly dictates otherwise.

As used herein, the term “addiction” broadly encompasses the process whereby physical and/or psychological dependence to a drug has developed. The withdrawal symptoms can reinforce the addiction, driving the user to continue taking the drug.

As used herein, the term “substance use disorder” also known as drug use disorder, is a condition in which the use of one or more substances leads to a clinically significant impairment or distress. The term “drug addiction” refers to a state of periodic or chronic intoxication produced by the repeated consumption of a drug (natural or synthetic).

As used herein, the term “physical dependence” (or “drug dependence”) refers to a state resulting from habitual use of a drug, where negative physical withdrawal symptoms result from abrupt discontinuation.

The term “biological sample” refers to a sample of tissue, cells, or fluid isolated from a subject, including, but not limited to, for example, blood, buffy coat, plasma, serum, blood cells (e.g., peripheral blood mononucleated cells (PBMCS), band cells, neutrophils, metamyelocytes, monocytes, or T cells)), fecal matter, urine, bone marrow, bile, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components.

The term “biomarker” refers to a protein which is present in a sample taken from subjects having substance use as compared to a comparable sample taken from control subjects. The biomarker can be a protein or a peptide fragment thereof.

A “control level” of a biomarker can be any amount or a range of an amount which is to be compared against a test amount of a biomarker.

As used herein, the term “early detection” of disorder refers to discovering the likelihood of substance use disorder before addiction.

As used herein, the term “prediction” refers to the likelihood that a patient will suffer a disorder described herein or that a patient will respond either favorably or unfavorably to a drug or set of drugs, and also the extent of those responses.

The term “subject” shall mean humans.

The term “susceptibility” refers to a constitution or condition of the body which makes the tissues react in special ways to certain extrinsic stimuli and thus tends to make the individual more than usually susceptible to certain diseases.

The term “risk” refers to the estimated chance of getting a disease during a certain time period, such as within the next 10 years, or during the lifetime.

The term “prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease.

The present disclosure surprisingly found that several biomarkers or combinations thereof are relevant to substance use disorders and/or infectious diseases and can be used as indicatives of these disorders or diseases. The biomarkers can be used for detecting or predicting a substance use disorder or infection or monitoring the progress of the substance use disorder or infection, and/or predicting treatment response or prognosis of substance use disorder or infection in a subject.

Biomarkers Associated with Substance-Use Disorders

The present disclosure surprisingly found that in a biological sample, the level of NFL alone or in combination with one or more CCL11, Nectin-4, MPO and/or ADAM10 can be used as indicators of various symptoms for evaluating a substance use disorder.

Neurofilaments (NF) are neuron-specific cytoskeletal proteins highly expressed in large caliber myelinated axons and involved in radial growth, stabilization and polarization of neural cells, enabling therefore effective high-velocity axonal conduction. Neurofilament light polypeptide, also known as neurofilament light chain (NFL), is a neurofilament protein that in humans is encoded by the NEFL gene. Clinically, NFL has been proposed as a biomarker of axonal damage and has been linked with brain injury or atrophy irrespective of the cause of the disorder, including inflammatory, neurodegenerative, traumatic, and cerebrovascular diseases as well as with physiological aging (Khalil et al. 2018, Neurofilaments as biomarkers in neurological disorders. Nat Rev Neurol. 14; 577-589). However, the relation between NFL and substance use disorder is unknown. The present disclosure firstly shows that biomarker NFL is indictive of neurotoxicity severity in subjects with substance use and the level of NFL is increased in the subjects at least 1.5 times higher than controls.

Preferably, the plasma or serum level of NFL is higher than 5 pg/ml. In a further embodiment, the expression level of NFL is higher than 7 pg/ml. In some embodiments, the expression level of NFL ranges from about 5 pg/ml to about 150 pg/ml, about 5 pg/ml to about 120 pg/ml, about 5 pg/ml to about 100 pg/ml or about 5 pg/ml to about 90 pg/ml. Particularly, the substance is alcohol or ketamine.

C-C motif chemokine 11 (CCL11) is also known as eosinophil chemotactic protein and eotaxin-1 is a protein that in humans is encoded by the CCL11 gene. This gene is encoded on three exons and is located on chromosome 17. CCL11 has been found that CCL11 is associated with psychiatric disorders such as schizophrenia, bipolar/autism spectrum disorder; major depression; dysthymia (Teixeira, A. L. et al. 2018, Revisiting the Role of Eotaxin-1/CCL11 in Psychiatric Disorders. Front Psychiatry 9: 241), and Alzheimer's disease (Morgan, A. R. et al. 2019, Inflammatory biomarkers in Alzheimer's disease plasma. Alzheimers Dement 15:776-787). Hsiang-Wei Kuo et al. suggested possible novel mechanisms mediated through CCL11 involving neurotoxicity in heroin dependent patients (Inflammatory chemokine eotaxin-1 is correlated with age in heroin dependent patients under methadone maintenance therapy. Drug Alcohol Depend, 2018; 183:19-24). The present disclosure firstly shows that biomarker CCL11 is indictive of chronic stress in a subject with substance use and the level of CC111 is increased in the subjects at least 1.0 times higher than controls.

Preferably, the blood level of CCL11 is higher than 40 pg/ml. In a further embodiment, the expression level of CCL11 is higher than 50 pg/ml, preferably 60 pg/ml, more preferably, 65 pg/ml. In some embodiments, the expression level of CCL11 ranges from about 40 pg/ml to about 100 pg/ml, about 50 pg/ml to about 100 pg/ml, about 60 pg/ml to about 100 pg/ml or about 65 pg/ml to about 100 pg/ml. Particularly, the substance is an opioid or ketamine.

Nectin Cell Adhesion Molecule 4 (Nectin-4) is a gene encoding an adhesion protein. Diseases associated with nectin-4 include Ectodermal Dysplasia-Syndactyly Syndrome 1 and Ectodermal Dysplasia. Among its related pathways are Sertoli-Sertoli Cell Junction Dynamics and Cell junction organization (https://www.genecards.org/cgi-bin/carddisp.pl?gene=NECTIN4). The present disclosure firstly shows that the secret form of biomarker nectin-4 is indictive of skin irritation in subjects with substance use and the level of nectin-4 is increased in the subjects at least 1.0 times higher than controls.

Preferably, the plasma or serum level of nectin-4 is higher than 50 pg/ml. In a further embodiment, the expression level of nectin-4 is higher than 60 pg/ml, preferably 70 pg/ml, more preferably, 80 pg/ml, 90 pg/ml, 100 pg/ml, 120 pg/ml, 130 pg/ml, 140 pg/ml or 150 pg/ml. In some embodiments, the expression level of nectin-4 ranges from about 50 pg/ml to about 250 pg/ml, about 60 pg/ml to about 250 pg/ml, about 70 pg/ml to about 250 pg/ml or about 70 pg/ml to about 150 pg/ml. Particularly, the substance is an opioid or ketamine.

Myeloperoxidase (MPO) is a peroxidase enzyme that in humans is encoded by the MPO gene on chromosome 17. MPO has been proposed as a biomarker of diabetes, cardiovascular diseases including coronary artery disease, congestive heart failure, arterial hypertension, pulmonary arterial hypertension, peripheral arterial disease, myocardial ischemia/reperfusion-related injury, stroke, cardiac arrhythmia and venous thrombosis (Ndrepepa, G., 2019, Myeloperoxidase—A bridge linking inflammation and oxidative stress with cardiovascular disease. Clin Chim Acta 493:36-51). However, the relation between MPO and substance use is unknown. The present disclosure firstly shows that biomarker MPO is increased in substance use subjects compared to healthy controls. Particularly, the biomarker MPO is indictive of alcohol dependence in subjects with substance use and the level of MPO is increased in the subjects at least 1.0 times higher than controls.

Preferably, the plasma or serum level of MPO is higher than 30 pg/ml. In a further embodiment, the plasma or serum level of MPO is higher than 40 pg/ml, preferably 50 pg/ml. In some embodiments, the plasma or serum level of MPO ranges from about 30 pg/ml to about 200 pg/ml, about 40 pg/ml to about 200 pg/ml, about 50 pg/ml to about 250 pg/ml, about 30 pg/ml to about 150 pg/ml, about 40 pg/ml to about 150 pg/ml or about 50 pg/ml to about 150 pg/ml. Particularly, the substance is an opioid or alcohol.

A Disintegrin and metalloproteinase domain-containing protein 10, also known as ADAM10 or CDw156 or CD156c is a protein that in humans is encoded by the ADAM10 gene. ADAM10 has been proposed as a biomarker of chronic kidney disease (Vinothkumar, G. et al., 2018, Therapeutic impact of rHuEPO on abnormal platelet APP, BACE 1, presenilin 1, ADAM 10 and AP expressions in chronic kidney disease patients with cognitive dysfunction like Alzheimer's disease: A pilot study. Biomed Pharmacother 104:211-222), rheumatoid arthritis (Isozaki, T. et al., 2017, A disintegrin and metalloproteinase (ADAM)-10 as a predictive factor for tocilizumab effectiveness in rheumatoid arthritis. Mod Rheumatol 27:782-786) and chronic venous disease (Serra, R. et al., 2017, From varices to venous ulceration: the story of chronic venous disease described by metalloproteinases. Int Wound J 14:233-240). However, the relation between ADAM10 and substance use disorder is unknown. The present disclosure firstly shows that biomarker ADAM10 is increased in substance use subjects compared to healthy controls. Particularly, the biomarker ADAM10 is indictive of synaptopathy in subjects with substance use and the level of ADAM-10 is increased in the subjects at least 1.0 times higher than controls.

Preferably, the plasma level of ADAM10 is higher than 15 ng/ml. In a further embodiment, the plasma or serum level of ADAM10 is higher than 20, 22, 24, or 25 ng/ml. In some embodiments, the plasma level of ADAM10 ranges from about 15 ng/ml to about 200 ng/ml, about 18 ng/ml to about 200 ng/ml, about 20 ng/ml to about 150 ng/ml, about 20 ng/ml to about 140 ng/ml, about 20 ng/ml to about 120 ng/ml or about 20 pg/ml to about 100 pg/ml. Particularly, the substance is an opioid or alcohol.

The present disclosure also provides a kit for detecting or predicting a substance use disorder and/or monitoring the progress of the substance use disorder, and/or predicting treatment response or prognosis of substance use disorder as described herein.

Biomarkers Associated with Infections Caused by Hepatitis Virus or Human Immunodeficiency Virus

C-X-C motif chemokine ligand 10 (CXCL10) also known as Interferon gamma-induced protein 10 (IP-10) or small-inducible cytokine B10 is an 8.7 kDa protein that in humans is encoded by the CXCL10 gene.

N-cadherin, also known as Cadherin-2 (CDH2) or neural cadherin (NCAD) is a protein that in humans is encoded by the CDH2 gene.

Interleukin 7 (IL-7) is a protein that in humans is encoded by the IL7 gene.

Caspase-10 is an enzyme that, in humans, is encoded by the CASP10 gene.

The present disclosure surprisingly found that the level of IP-10 alone or in combination with one or more CDH2, IL-7 and/or Caspase-10 can be used an indictor for evaluating infectious diseases and provides advantageous detection of an infection caused by hepatitis virus or human immunodeficiency virus. The level of the above biomarkers in a biological sample from a subject that is higher than that of a control is indicative of a substance use disorder.

Particularly, the plasma level of IP-10 is higher than 510 pg/ml. In a further embodiment, the expression level of IP-10 is higher than 550 pg/ml, 600 pg/ml, 650 pg/ml, 700 pg/ml, 750 pg/ml, 800 pg/ml, 850 pg/ml, 900 pg/ml, 950 pg/ml, 1,000 pg/ml, 1,050 pg/ml, or 1,100 pg/ml.

Particularly, the plasma expression level of CDH2 is higher than 10 pg/ml. In a further embodiment, the expression level of CDH2 is higher than 12 pg/ml or 14 pg/ml.

In a further embodiment, the plasma level of IL-7 is higher than 4 pg/ml. In a further embodiment, the expression level of IL-7 is higher than 5 pg/ml.

The present disclosure also provides a kit for detecting or predicting an infection caused by hepatitis virus or human immunodeficiency virus and/or monitoring the progress of the infection, and/or predicting treatment response or prognosis of the infection as described herein.

Detection of Biomarkers

Development of a detection or diagnostic test for a substance use disorder or an infection starts with the collection of a biological sample from a subject to isolate and capture an active form of the peptide or protein fragment of the biomarker described herein, followed by the generation of antibodies against this peptide/protein fragment. Once the antibodies have been generated, the next step is testing the antibody with the biological sample from subjects relative to normal control biological samples. Any conventional immunoassay format may be used. Examples of the immunoassay include, but are not limited to, the following.

Immunofluorescent assay is a technique in which specific antibodies are tagged with a fluorescent dye. When these antibodies bind to the protein or peptide of the biomarker described herein, they appear as fluorescent, glowing particles under a fluorescent microscope, or are measured in a fluorescent spectrometer, thereby revealing their location, particularly when bound to the peptide as an indicator of the peptide being present in the sample. Likewise, the peptide may be tagged with a fluorescent dye for the opposite effect. The tagged peptide may be either the sample peptide or a previously synthesized peptide.

Enzyme immunoassays uses tagging with an enzyme. An enzyme substrate or product is a readily detectable substance wherein measurement of the formation or reduction of the readily detectable substances determines the presence of the antibody or peptide tagged. Typically, a developing solution is added that the enzyme catalyzes to form a color change. Thus a simple viewing of the color determine whether or not the sample comes from a patient with SJS. Of particular interest is an Enzyme-linked immunosorbent assay (ELISA). In this format the antibody is first bound to a solid phase such as the inside of a container or on a small particle. The sample containing a peptide is added and allowed to bind to the antibody. A tagged second specific antibody is then added which binds to the peptide so that the peptide is “sandwiched” between the antibodies. Optionally, a developing solution is added for the tag to react with and the result is measured or observed. Presence of the readily detectable signal indicates presence of the peptide in the sample. Alternatively, the binding of antibody to solid phase may be performed later in the assay and the antibody-peptide binding may be performed before adding it to a container.

Radioimmunoassay (RIA) is a similar technique to an immunofluorescence assay that tags an antibody or peptide with radioactive material. Detection is typically performed by a scintillation counter.

In some embodiments, a detection test to correctly predict status is measured as the sensitivity of the assay, the specificity of the assay or the area under a receiver operated characteristic (ROC) curve (AUC). The greater the area under the ROC curve, for example, the more accurate or powerful the predictive value of the test.

In some embodiments, one or more of the biomarkers disclosed herein show a statistical difference in different samples of at least p<0.05. Detection tests that use these biomarkers may show an AUC of at least 0.9.

Although the present disclosure has been described with an exemplary embodiment, various changes and modifications may be suggested to one skilled in the art. It is intended that the present disclosure encompassed such changes and modifications as fall within the scope of the appended claims.

### EXAMPLE

The other characteristics and advantages of the present invention are further illustrated and described in the following examples. The examples described herein are used for the purpose of illustration, and do not limit the present disclosure.

The practice of the present invention will employ technologies comprising conventional techniques of cell biology and cell culture, which are within the ordinary skills of the art. Such techniques are explained fully in the literature.

Subject Population

The experiment protocol is approved by the institutional review boards of the National Health Research Institutes (EC0970504, Zhunan, Taiwan) and the six participating hospitals, Tao-Yuan Mental Hospital, En-Chu-Kong Hospital, Far-Eastern Memorial Hospital, Taipei City Hospital Song-De and Yang-Ming Branches, China Medical University Hospital, and Wei-Gong Memorial Hospital. Written informed consents are obtained from all participants. The project has also been registered with the National Institutes of Health (NIH) Clinical Trial (https://clinicaltrials.gov/ct2/show/NCT01059747 for 344 MMT patients). The inclusion criteria included subjects aged 18 or above, having undergone MMT for at least three months with regular attendance for the past seven days, and a methadone dosage adjustment of not more than 10 mg in the past seven days. Exclusion criteria included co-morbidity with physical and mental disorders requiring immediate treatment and pregnancy.

The protocol for comparing the normal control with MMT study and former heroin user subjects is approved by the Institutional Review Board of the National Health Research Institutes (EC0980209-R5, Zhunan, Taiwan).

Cytokines Evaluation

The concentration of inflammatory cytokines and chemokines (CCL11 and IP-10) are determined in human blood samples by using the Milliplex MAP human cytokine/chemokine magnetic bead panel kit (Millipore, Billerica, Mass.). The analyses are performed according to the protocol of the manufacture. All sample data are acquired from a MAGPIX Multiplex Reader (Luminex Corp., Austin, Tex.). Additionally, a quantitative sandwich enzyme immunoassay technique (R&D Systems, Minneapolis, Minn.) is used to detect soluble nectin-4 in plasma of patients, and the caspase 10 is measured by enzyme-linked immuno-sorbent assay (ELISA) (Bioassay technology laboratory, Shanghai, China). On the other hand, the plasma level of Cadherin 2 is measured by the enzyme-linked immuno-sorbent assay (ELISA) kits according to the manufacture's instructions (Cusabio Biotech (Wuhan, China), the plasma levels of ADAM10 were measured by the enzyme-linked immuno-sorbent assay (ELISA) kits according to the manufacture instructions from Elabscience Biotechnology (Wuhan, China). And the serum neurofilament light polypeptide concentration is measured by using a ‘single molecule array’ (SIMOA) ELISA assay.

### Example 1. Screening HIV and/or HCV Indication Biomarkers in Addicted Subjects

There are a total of 331 subjects screened for HCV and 329 screened for HIV antibody. A detailed demography of the subjects is shown in Table 1 below. Only one patient wasexcluded from further analysis for testing positive for HIV-antibody but not HCV. Higher levels of AST (P=0.048) and ALT (P=0.046) are found in the HIV(+)/HCV(+) or HCV(+) patients than the HIV(−)/HCV(−) patients (Table 1). The plasma level of IP-10 is highest in the MMT patients co-infected with HIV and HCV, followed by those infected with HCV only, then those without any infection (Table 1). In the analyses to test the correlation between plasma levels of chemokine/cytokines and liver functional parameters, IP-10 level shows significant correlations with levels of AST (r=0.294, P<0.0001), ALT (r=0.232, P<0.0001) and γ-GT (r=0.247, P<0.0001) in both positive and negative urine morphine test patients.

TABLE 1
Demography of subjects under methadone treatment and their HIV/HCV infection status.

| | Overall | | HIV(+)/HCV (+) | | HCV (+) | | HIV(−)/HCV (−) | | |
|---|---|---|---|---|---|---|---|---|---|
| Variable | N | Mean ± SD | N | Mean ± SD | N | Mean ± SD | N | Mean ± SD | P-value |
| Age (years) | 331 | 38.26 ± 7.71 | 73 | 37.36 ± 7.86 | 238 | 38.6 ± 7.77 | 17 | 36.76 ± 6.31 | 0.37 |
| (Minimum~Maximum) | (24~66) | | (24~63) | | (25~66) | | (25~48) | | |
| Male | 272 | (82.18%) | 62 | (84.93%) | 192 | (80.67%) | 16 | (94.12%) | 0.30 |
| Liver Function | | | | | | | | | |
| AST, U/L | 317 | 52.77 ± 57.79 | 69 | 57.1 ± 51.33 | 229 | 53.23 ± 61.56 | 17 | 31.35 ± 12.08 | 0.048* |
| ALT, U/L | 323 | 61.48 ± 76.19 | 73 | 50.52 ± 42.81 | 231 | 66.94 ± 85.89 | 17 | 37.59 ± 28.70 | 0.046* |
| CXC chemokines | | | | | | | | | |
| IP-10, pg/ml | 327 | 1162.35 ± 806.54 | 73 | 1561.78 ± 906.07 | 234 | 1079.43 ± 730.94 | 17 | 512.84 ± 315.85 | <0.0001* |

*Significant difference

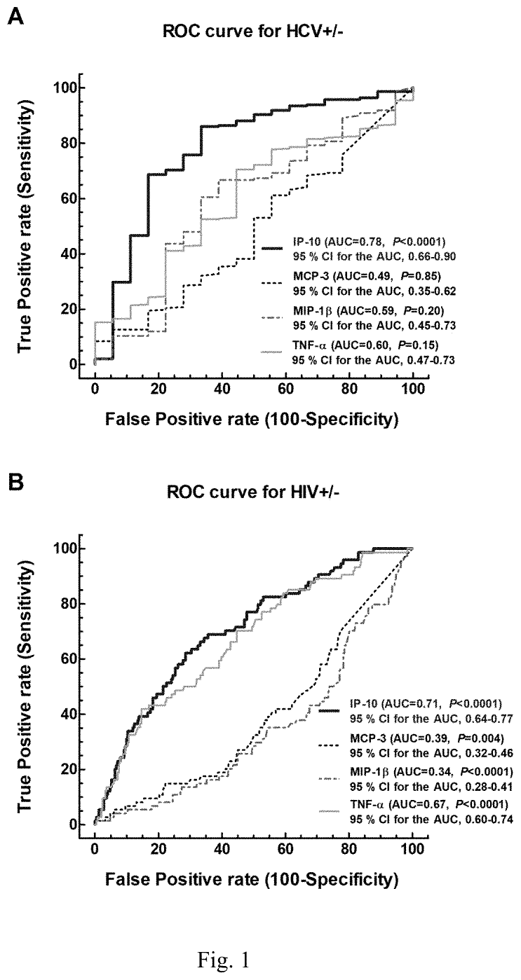

As shown in FIG. 1, the area under the curve (AUC)/receiver operating characteristic (ROC) analyses shows that the level of IP-10 has the largest AUC to predict both HCV and HIV infection (AUC=0.78; 95% CI 0.66-0.90; P<0.0001 and AUC=0.71; 95% CI 0.64-0.77; P<0.0001). This result indicates that IP-10 is a potential biomarker for the prediction of HCV and HIV infection. In contrast, the level of TNF-α has a larger AUC (0.67; 95% CI 0.60-0.74; P<0.0001) relating to the status of HIV infection only.

On the other hand, it is also found that the plasma level of CDH2 is correlated with the status of HIV infection, plasma level of cytokine IL-7, and treatment outcome. In multivariate regression analyses, plasma CDH2 level is significantly associated with plasma level of cytokine IL-7(β=−0.32, P=0.0008), HIV infection status (β=−5.35, P=0.003), and the urine morphine test results (β=−3.24, P=0.028) (Table 2a). In further subgrouping analyses, the HIV positive MMT patients have a lower plasma CDH2 level than the HIV negative patients (GLM, permutation P=0.009) (Table 2b). Subjects with a higher plasma CDH2 level show a better treatment outcome (GLM, permutation P=0.006).

TABLE 2a
Multivariate regression analyses of the plasma CDH2 level (ng/ml)

| | | | P- | Adjust- | Partial | |
|---|---|---|---|---|---|---|
| Variable^{a} | β | S.E. | value^{b} | ed^{c} | r² | VIF |
| Plasma IL-7 (pg/ml) | −0.32 | 0.09 | 0.0008 | 0.0009 | 0.036 | 1.03 |
| HIV (+/−) | −5.35 | 1.73 | 0.003 | 0.002 | 0.032 | 1.04 |
| Urine morphine (+/−) | −3.24 | 1.47 | 0.028 | 0.039 | 0.012 | 1.04 |
| Cotinine | 0.01 | 0.004 | 0.06 | 0.07 | 0.012 | 1.03 |
| Concentration (ng/ml) | | | | | | |
| Heart rate (beats/min) | 0.11 | 0.06 | 0.07 | 0.06 | 0.013 | 1.05 |
| Systolic blood | 0.05 | 0.04 | 0.2 | 0.29 | 0.005 | 1.04 |
| pressure (mmHg) | | | | | | |

TABLE 2b
Plasma levels of CDH2 and IL-7 were associated with HIV
infection status and the urine morphine test outcome.

| | Plasma CDH2 level (ng/ml) | | | Plasma IL-7 level (pg/ml) | | |
|---|---|---|---|---|---|---|
| Variable | n | Mean ± SD | P-value | n | Mean ± SD | P-value |
| HIV | | | 0.009 ^{d} | | | 0.013 ^{d} |
| HIV (+) | 76 | 12.97 ± 10.79 | 0.008 ^{c} | 76 | 4.59 ± 5.77 | 0.045 ^{c} |
| HIV (−) | 261 | 17.41 ± 13.64 | | 257 | 7.31 ± 8.83 | |
| Urine morphine test | | | 0.006 ^{d} | | | 0.31 ^{d} |
| Morphine (+) | 173 | 14.59 ± 10.82 | 0.005 ^{c} | 170 | 7.14 ± 8.69 | 0.38 ^{c} |
| Morphine (−) | 169 | 18.49 ± 14.92 | | 167 | 6.23 ± 7.80 | |

HIV, human immunodeficiency virus; β, stepwise regression coefficient; S.E., standard error of regression coefficient; VIF, variance inflation factor; SD, standard deviation ^{a}n=319, F=6.38, P<0.0001, adjusted r²=9.24%.^{b}permutation P-Value. ^{c}adjusted for all other taken medications.^{d}General linear module of permutation P-value.

### Example 2. Evaluating the correlation between and the level of Caspase-10 Protein

13 patients who had no HBsAg data and 3 patients who had received medications for HIV treatment, which may cause drug-drug interactions, were excluded. Thus, data from a total of 328 patients were analyzed in this study (Table 3).

TABLE 3
Comparison of general demography between HBsAg positive
and negative patients in methadone maintenance treatment.

| Variables | HBsAg (+) | HBsAg (−) | P-value |
|---|---|---|---|
| Age (years) | | | |
| n, Mean ± SD | 75, | 253, | 0.54^{a} |
| | 37.8 ± 7.6 | 38.4 ± 7.7 | |
| Gender | | | |
| Male | 67 (89.33%) | 201 (79.45%) | 0.052^{b} |
| Female | 8 (10.67%) | 52 (20.55%) | |
| Caspase-10 (ng/ml) | | | <0.0001^{a} |
| n, Mean ± SD | 68, | 239, | |
| | 3.5 ± 1.7 | 11.6 ± 12.9 | |

HBsAg, hepatitis B surface antigen; BMI, body mass index;
^{a}Mann-Whitney U test;
^{b}Chi-square test.

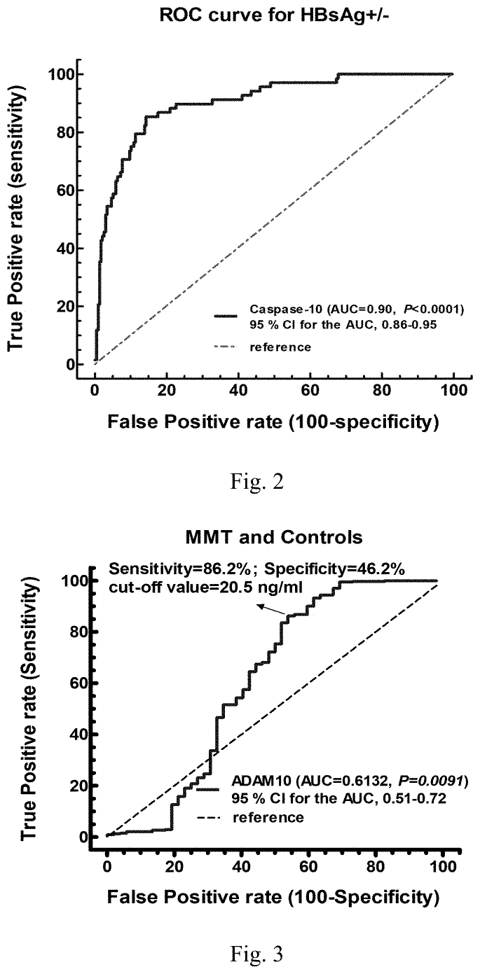

To further characterize the role of caspase-10 in HBV infection, a receiver operating characteristic curve (ROC) was performed between the HBsAg (+) and (−) patients. Level of caspase-10 showed a strong predictive effect in both sensitivity and specificity for HBsAg with area under the curve (AUC)=0.90, 95% CI 0.86-0.95, P<0.0001 in FIG. 2.

### Example 3. Peripheral Biomarker ADAM10 is Increased in MTT Patients and Correlated with the Levels of Metabolites of Vitamin D, Methadone, and Nicotine

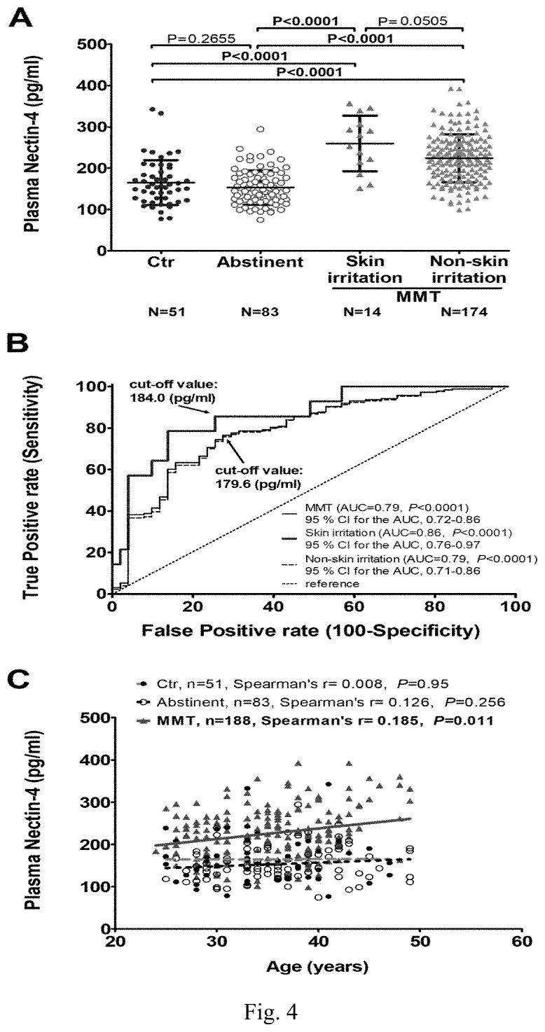

344 MMT patients and 52 controls in the study. Detailed demography of subjects is shown in below Table 4. The age of patients with MMT was 38.2±7.7 and the controls was 36.4±7.2. Gender was significantly difference between MMT and controls (P=0.0009). Higher levels of plasma ADAM10 (P=0.009) in the MMT than controls. FIG. 3 shows ROC curve analyses of plasma levels of ADAM10 with MMT patients and controls

TABLE 4
General demographic and clinical characteristics
in MMT patients and controls

| | Controls | MMT patients | |
|---|---|---|---|
| Variables | (N = 52) | (N = 344) | P-value |
| Age, years, mean ± SD | 36.4 ± 7.2 | 38.2 ± 7.7 | 0.11 |
| Gender, N (%) | | | 0.0009 |
| Male | 32 (61.5) | 281 (81.7) | |
| Female | 20 (38.5) | 63 (18.3) | |
| ADAM10 (ng/ml) | 23.7 ± 11.7 | 26.9 ± 6.3 | 0.009 |

Mann-Whitney U test used for continuous variables; Chi-square test used for gender.

The plasma level of ADAM10 was negatively associated with plasma levels of IL-7 and 25-hydroxy vitamin D, and positively associated with the levels of cotinine concentration, R-methadone, S-methadone and (R, S) methadone, and dose change. Using univariate regression analyses with permutations, The plasma level of ADAM10 was correlated with dose change (P=0.0004), plasma levels of IL-7 (P=0.01), R-Methadone (P=0.0004), S-Methadone (P=0.003), (R, 5) methadone (P=0.0004), cotinine concentration (P=0.002) and 25-hydroxy vitamin D (P=0.0004) (Table 5).

TABLE 5
Univariate regression analyses ADAM10 and methadone treatment responses.

| | | | | |
|---|---|---|---|---|
| Variable | n | β | P-value | Adjusted |

Plasma ADAM10 (ng/ml)
General

| | | | | |
|---|---|---|---|---|
| Maintenance methadone dosage(mg/day) | 341 | 0.026 | 0.028 | 0.019 |
| Dose change (Tolerance) | 330 | 0.039 | 0.0004 | 0.0003 |
| Plasma CDH2 (ng/ml) | 341 | 0.047 | 0.07 | 0.08 |
| Plasma IL-7 (pg/ml) | 337 | −0.11 | 0.010 | 0.010 |

Plasma concentrations of methadone and its metabolites

| | | | | |
|---|---|---|---|---|
| R-Methadone (ng/ml) | 341 | 0.01 | 0.0004 | 0.0002 |
| S-Methadone (ng/ml) | 341 | 0.01 | 0.003 | 0.002 |
| S-Methadone/methadone dose ratio | 341 | 0.49 | 0.029 | 0.030 |
| (R,S) methadone (ng/ml) | 341 | 0.006 | 0.0004 | 0.0005 |
| Cotinine concentration (ng/ml) | 341 | 0.006 | 0.002 | 0.001 |
| 25-hydroxy vitamin D (nM) | 341 | −0.045 | 0.0004 | 0.0003 |

Cardiovascular

| | | | | |
|---|---|---|---|---|
| Systolic blood pressure (mmHg) | 328 | 0.036 | 0.06 | 0.054 |
| Heart rate score | 340 | 1.24 | 0.040 | 0.045 |

β, regression coefficient.
P-value, permutation P-value.
Adjusted, permutation P-value adjusted for all other taken medications.

A multivariate regression analysis showed that plasma level of ADAM10 was associated with the plasma levels of 25-hydroxy vitamin D(β=−0.04, P=0.002), nicotine metabolite cotinine (β=0.01, P=0.006), and R-methadone (β=0.01, P=0.004) (Table 6).

TABLE 6
Multivariate regression analyses of
the plasma ADAM10 level (ng/ml).

| | | | P- | Adjust- | Partial | |
|---|---|---|---|---|---|---|
| Variable | β | S.E. | value | ed | r² | VIF |
| 25-hydroxy | −0.04 | 0.012 | 0.002 | 0.001 | 0.047 | 1.06 |
| vitamin D (nM) | | | | | | |
| Cotinine | 0.01 | 0.002 | 0.006 | 0.006 | 0.028 | 1.02 |
| Concentration (ng/ml) | | | | | | |
| R-Methadone (ng/ml) | 0.01 | 0.003 | 0.004 | 0.002 | 0.025 | 1.03 |
| Plasma IL-7 (pg/ml) | −0.07 | 0.041 | 0.08 | 0.09 | 0.011 | 1.05 |
| Systolic blood | 0.02 | 0.019 | 0.22 | 0.27 | 0.005 | 1.02 |
| pressure (mmHg) | | | | | | |
| Plasma CDH2 (ng/ml) | 0.02 | 0.026 | 0.49 | 0.47 | 0.001 | 1.07 |

n = 325, F = 6.97, P < 0.0001, adjusted r² = 9.99%.
β, stepwise regression coefficient.
S.E., standard error of regression coefficient.
P-value, permutation P-value.
VIF, variance inflation factor.
Adjusted, permutation P-value adjusted for all other taken medications.

### Example 4. Evaluating the Skin Irritation in Methadone Maintenance Treatment Patients

The enrolled 344 MMT patients had an average age of 38 years and 81.7% were male. The average methadone dosage was 55.22 mg/day. Concentration of R,S-methadone in plasma was 336.96 ng/ml. Plasma level of cytokine TNF-α was 10.55 pg/ml. Plasma nectin-4 concentration was 238.31 pg/ml. Fifteen patients (around 4.4% in 344 MMT patients) self-reported with skin irritation after taking methadone (as shown in Table 7).

TABLE 7
General demography of the methadone maintenance
treatment patients in this study

| Variable | n | Mean ± SD |
|---|---|---|
| Age (year) | 344 | 38.16 ± 7.69 |
| BMI | 341 | 23.64 ± 3.52 |
| Gender | | |
| Male | 281 | (81.69%) |
| Female | 63 | (18.31%) |
| Skin Irritation of side effect | 15 | 1.47 ± 0.83 |
| Methadone dosage (mg/day) | 344 | 55.22 ± 28.47 |
| R-Methadone (ng/ml) | 344 | 194.44 ± 123.56 |
| S-Methadone (ng/ml) | 344 | 142.52 ± 99.64 |
| R,S-methadone (ng/ml) | 344 | 336.96 ± 212.72 |
| TNF-α (pg/ml) | 339 | 10.55 ± 8.68 |
| Nectin-4 (pg/ml) | 311 | 238.31 ± 79.64 |

SD, Standard deviation.

Therefore, the plasma nectin-4 levels among age- and gender-matched controls (±3 years), medication-free abstinent former heroin users, and MMT patients are further examined. As shown in Table 8, the abstinent former heroin users have a higher average BMI than MMT subjects and controls. The rates for HCV infection are 100% and 92% respectively in MMT patients with and without skin irritation, 80% in former heroin users, and 0% in the controls.

TABLE 8
General demography between age- gender- matched control, medication-free abstinent former heroin
users (abstinent), MMT patients with skin irritation, and MMT patients without skin irritation

| | | | Medication-free | | MMT patients | | | | | |
|---|---|---|---|---|---|---|---|---|---|---|
| | Control ¹ | | abstinent ² | | Skin irritation ³ | | No skin irritation ⁴ | | | |
| Variable | N | Mean ± SD | N | Mean ± SD | N | Mean ± SD | N | Mean ± SD | P-value | Post hoc |
| Age | 51 | 34.14 ± 6.05 | 83 | 35.87 ± 6.00 | 15 | 38.27 ± 7.35 | 174 | 35.36 ± 5.60 | 0.12 | |
| BMI | 51 | 24.60 ± 3.89 | 82 | 26.28 ± 3.44 | 15 | 21.99 ± 2.82 | 173 | 23.78 ± 3.16 | <0.0001 | 2 > 1, 3, 4**; |
| | | | | | | | | | | 1, 4 > 3* |
| Gender | | | | | | | | | 0.09 | |
| Male | 43 | (84.31%) | 80 | (96.39%) | 14 | (93.33%) | 153 | (87.93%) | | |
| Female | 8 | (15.69%) | 3 | (3.61%) | 1 | (6.67%) | 21 | (12.07%) | | |
| Nectin-4 (pg/ml) | 51 | 164.91 ± 54.54 | 83 | 153.23 ± 41.71 | 14 | 259.67 ± 67.48 | 174 | 224.03 ± 58.36 | <0.0001 | 3, 4 > 1, 2** |
| HIV | | | | | | | | | <0.0001 | 3, 4 > 1**; 4 > 2*; |
| Negative | 50 | (100.00%) | 75 | (90.36%) | 11 | (78.57%) | 128 | (74.42%) | | 2 > 1* |
| Positive | 0 | (0.00%) | 8 | (9.64%) | 3 | (21.4%) | 44 | (25.58%) | | |
| HCV | | | | | | | | | <0.0001 | 2, 3, 4 > 1**; |
| Negative | 49 | (100.00%) | 16 | (19.51%) | 0 | (0.00%) | 14 | (8.33%) | | 4 > 2* |
| Positive | 0 | (0.00%) | 66 | (80.49%) | 13 | (100.00%) | 154 | (91.67%) | | |

Continuous variable test by Kruskal Wallis test for four groups and Mann-Whitney U test for multiple comparison (post hoc); the categorical variable analysis by Chi-square test.
AST, Aspartate aminotransferase.
ALT, Alanine aminotransferase.
γ-GT, Gamma-glutamyl transpeptidase.
HIV, Human Immunodeficiency Virus.
HCV, Hepatitis C virus antibody.
*P < 0.05;
**P < 0.01

Additionally, the plasma levels of nectin-4 of different group, MMT subjects with skin irritation, MMT subjects without skin irritation, control subjects, and former heroin users, are further examined (as shown in FIG. 4A). The rank of plasma levels of nectin-4 is as follow: MMT patients with skin irritation>MMT patients without skin irritation (P=0.051)>controls (P<0.0001)=former heroin users (P<0.0001). The plasma levels of nectin-4 at the cut-off value 184 (pg/ml) in controls and MMT subjects with skin irritation show the highest level of sensitivity (85.7%) and specificity (74.5%) (AUC=0.86, P<0.0001) as compared with MMT subjects without skin irritation and MMT patients in the ROC curve analyses (FIG. 4B). The plasma nectin-4 levels show a positive correlation with age (r=0.265, P<0.0001) and the addiction duration (r=0.259, P<0.0001) in all 344 MMT subjects. The association with age is observed only in MMT subjects (r=0.185, P=0.011) but not in the age- and gender-matched controls, or methadone-free abstinent former heroin users (FIG. 4C).

### Example 5. Examining the Influence of Age on Plasma CCL11 Level

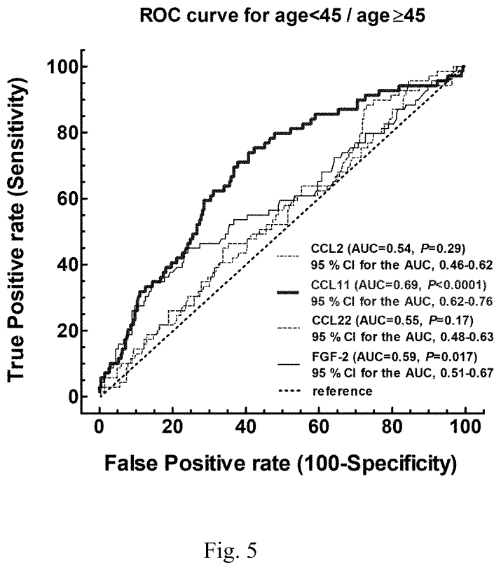

Comparing the levels of correlation between the concentrations of chemokine ligands, FGF-2 and age, CCL11 at 73.5 (pg/ml) shows the highest level of sensitivity (71%) and specificity (62%) in correlation with age (AUC=0.69, P<0.0001) as compared with the FGF-2 (AUC=0.59, P=0.017) (FIG. 5). The best cut-off age for CCL11 was 45 years old (Table 9). The average levels of plasma CCL11 in younger subjects is 68.76±30.47 pg/ml; however, in subjects older than 45 years old, the average plasma CCL11 level is 91.14±41.25 pg/ml (Table 10). Additionally, subgrouping these subjects by age, the cut-off value of 45 year-old, the percentage of male (P<0.0001), the plasma nicotine metabolite cotinine concentration (P=0.022), the addiction duration (P<0.0001), the plasma level of CCL11 (P<0.0001) and plasma level of FGF-2 (P=0.017) are all significantly higher in subjects older than 45 years old.

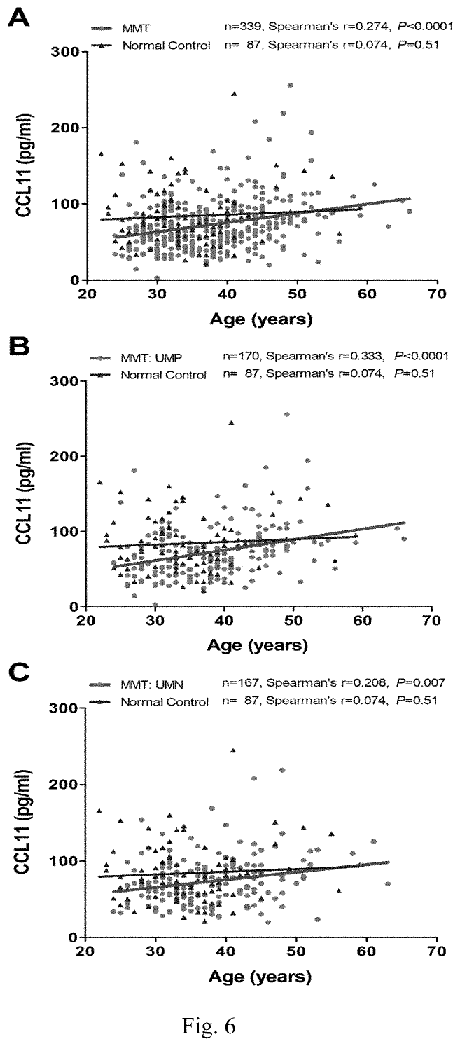

Therefore, linear correlation analysis is used to evaluate the correlation between plasma CCL11 level and age in both MMT subjects and normal subjects. The results indicate that there is a significant correlation (r=0.27, slope=1.21, P<0.0001) between plasma CCL11 levels and age in the MMT subjects instead of the normal controls (r=0.074, slope=0.36, P=0.51) (FIG. 6A). When further comparing the subgroup of MMT subjects based on treatment response by the urine morphine test results, the urine morphine test positive subjects (r=0.33, slope=1.4, P<0.0001) (FIG. 6B) show a greater slope of correlation between the plasma CCL11 levels and age than the urine morphine test negative subjects (r=0.21, slope=0.99, P=0.007) (FIG. 6C).

TABLE 9
The receiver operating characteristic (ROC)
curve identified the best cut-off age.

| Age | | | | |
|---|---|---|---|---|
| cut-off | The number of less than | | | |
| value | the age cut-off value | AUC | 95% C.I. | P-value |
| 30 | 43 | 0.582 | 0.49-0.68 | 0.09 |
| 35 | 122 | 0.587 | 0.52-0.65 | 0.008 |
| 40 | 206 | 0.668 | 0.61-0.73 | <0.0001 |
| 45 | 275 | 0.688 | 0.62-0.76 | <0.0001 |
| 50 | 319 | 0.694 | 0.59-0.80 | 0.001 |

AUC = Area under the curve; C.I. = Confidence interval.

TABLE 10
Comparison of the variables between subjects
older and younger than 45 years in MMT study.

| | age <45 | | age ≥45 | | |
|---|---|---|---|---|---|
| Variable | n | Mean ± SD | n | Mean ± SD | P-value ^{a} |
| Gender | | | | | <0.0001^{b} |
| Male | 213 | (77.45%) | 68 | (98.55%) | |
| Female | 62 | (22.55%) | 1 | (1.45%) | |
| BMI (kg/m²) | 272 | 23.72 ± 3.55 | 69 | 23.29 ± 3.39 | 0.39 |
| Cotinine (ng/ml) | 275 | 384.20 ± 184.90 | 69 | 439.65 ± 203.44 | 0.022 |
| Addiction duration (year) | 275 | 11.22 ± 5.78 | 69 | 20.01 ± 9.31 | <0.0001 |
| CCL2 (pg/ml) | 270 | 219.60 ± 150.26 | 69 | 236.83 ± 126.12 | 0.29 |
| CCL11 (pg/ml) | 270 | 68.76 ± 30.47 | 69 | 91.14 ± 41.25 | <0.0001 |
| CCL22 (pg/ml) | 270 | 697.40 ± 294.97 | 69 | 755.82 ± 302.28 | 0.17 |
| FGF-2 (pg/ml) | 270 | 89.42 ± 66.60 | 69 | 130.55 ± 137.81 | 0.017 |

^{a} Wilcoxon rank-sum test.
^{b}Chi-square test.

Furthermore, further analyses of major factors influencing the plasma CCL11 concentrations are conducted by using multivariate regression analyses. The results reveal that FGF-2 (partial r²=0.24, β=0.17, 95%, confidence interval (CI)=(0.14-0.21), P<0.0001) shows the strongest positive correlations with plasma CCL11 concentrations, as compared with age (partial r²=0.069, β=0.74, 95% CI=(0.35-1.12), P=0.0006)), CCL2 (partial r²=0.036, β=0.046, 95% CI=(0.026-0.067), P=0.005), and CCL22 (partial r²=0.022 , β=0.012, 95% CI=(0.001-0.022), P=0.028) concentrations, followed by plasma cotinine concentration (partial r²=0.013, β=0.02, 95% CI=(0.005-0.036), P=0.01) (Table 11)

TABLE 11
Multiple regression analyses of the plasma level of CCL11 (pg/ml).

| Variable | β | 95% CI | S.E. | t | P-value ^{per} | VIF | Partial R² |
|---|---|---|---|---|---|---|---|
| Age | 0.74 | (0.35-1.12) | 0.20 | 3.78 | 0.0006 | 1.05 | 0.069 |
| Cotinine (ng/ml) | 0.020 | (0.005-0.036) | 0.008 | 2.60 | 0.010 | 1.02 | 0.013 |
| CCL2 (pg/ml) | 0.046 | (0.026-0.067) | 0.011 | 4.39 | 0.005 | 1.09 | 0.036 |
| CCL22 (pg/ml) | 0.012 | (0.001-0.022) | 0.005 | 2.25 | 0.028 | 1.12 | 0.022 |
| FGF-2 (pg/ml) | 0.17 | (0.14-0.21) | 0.02 | 9.89 | <0.0001 | 1.09 | 0.239 |

β = regression coefficient; CI = Confidence interval; S.E. = standard error of regression coefficient; ^{per} = Permutation test; VIF = variance inflation factor.
F = 40.64,
P < 0.0001,
adjusted R² = 36.96%,
n = 339

### Example 6. Peripheral Biomarker NFL is Increased in Ketamine-Dependent Patients

Participants

This cross-sectional study was conducted at Taipei City Psychiatric Center (TCPC), Taipei, Taiwan after approval from Research Ethics Committee (no. TCHIRB-1030408) was obtained. Treatment-seeking patients with ketamine dependence were consecutively screened for the eligibility for participating in this study from Department of Addiction Sciences of TCPC. The inclusion criteria were as follows: (1) age between 18 and 60 years; (2) fulfilling the Diagnostic and Statistical Manual of Mental Disorders, 4th Edition, Text Revision (DSM-IV-TR) criteria for ketamine dependence, as verified by two board-certified psychiatrists; (3) most recent ketamine use within 24 hours prior to admission, validated by a self-report and positive test for ketamine in urine toxicology; and (4) ability to read Chinese and provide informed consent. The exclusion criteria were (1) presence of another substance use disorder (including abuse and dependence) based on clinical interview and urine tests during the preceding year, except for nicotine; (2) history of schizophrenia, or bipolar disorder, or treatment with antipsychotics or mood stabilizers (including lithium, valproic acid, carbamazepine, and quetiapine); (3) history of systemic illnesses such as hypertension, metabolic disorders (e.g., diabetes mellitus), or severe renal or liver disease; (4) history of head injury, loss of consciousness, or neurological disorders; and (5) inability or refusal to provide a urine sample. Healthy controls were enrolled from the physical check-up unit at the hospital, with the following inclusion criteria: (1) age between 18 and 60 years; (2) no substance use disorder (including abuse and dependence) during the preceding year, except for nicotine; (3) no history of a major psychiatric disorder (including schizophrenia, schizoaffective disorder, bipolar disorder, major depressive disorder, and organic mental disorders) based on screening by a trained research assistant with a bachelor's degree in psychology using the Mini-International Neuropsychiatric Interview; (4) no known systemic diseases such as hypertension, metabolic disorders (e.g., diabetes mellitus), and renal and liver disease; (5) no history of head injury, loss of consciousness, or neurological disorders; and (5) ability to read Chinese and provide informed consent.

Procedure

After initial assessments, eligible participants were given a comprehensive description of the study and were then included after providing written informed consent for participation. A trained research assistant interviewed the participants via the Chinese version of the Diagnostic Interview for Genetic Studies (DIGS-C) and collected basic demographic data and a lifetime diagnosis of major depressive disorder (MDD). Average and maximum daily doses of ketamine and the number of ketamine use days during the last 30 days were recorded. Craving severity was measured using a Visual Analogue Scale (VAS), where participants self-rated their level of craving for ketamine following a careful explanation by the research assistant. Each patient indicated a position along a continuous line between 0 and 100, with 0 denoting no craving and 100 denoting a craving so severe that the individual was unable to resist ketamine if it were available. In addition, history of childhood trauma was assessed by the Childhood Trauma Questionnaire-short form (CTQ-SF). The CTQ-SF encompasses five types of childhood trauma, including emotional, physical, and sexual abuse, and emotional and physical neglect, each of them being measured based on five items and rated on a five-point Likert scale. Scores exceeding the cutoff point for moderate severity on each subscale (emotional abuse: ≥13; physical abuse: ≥10; sexual abuse: ≥8; emotional neglect: ≥15; physical neglect: ≥10) were indicated as positive for that type of childhood trauma. Total scores of CTQ-SF and number of types of childhood trauma were recorded. The Chinese-version of CTQ-SF has also been validated and showed favorable factor structure and test-retest reliability.

Assays

Venous blood samples were collected following overnight fasting between 8:00 and 9:00 a.m. for ketamine-dependent patients (on the second day of admission) and control participants. Serum NFL levels were determined at the Cold Spring Biotech Corp. (New Taipei City, Taiwan, R.O.C.) using an SiMoA platform provided by Quanterix (Quanterix Corporation, Billerica, Mass.). Measurements were performed on the fully automated instrument HD-1 Analyzer (Quanterix) using the NF-LIGHT® kit from Quanterix, which employs an anti-NFL monoclonal antibody produced by UmanDiagnostics (Umeå, Sweden). All specimens were assayed in duplicate, with coefficients of variance (CVs) between duplicate measurements below 20%. The limit of detection (mean blank signal+2.5 SD) for the SiMoA NFL assay was 0.11 pg/mL and the lower limit of quantification was 0.174 pg/mL when compensated for a four-fold sample dilution. For serum, the mean intra-assay coefficient of variation for duplicate determinations was 5.8%. The staff performing the analyses was blinded to all phenotype information.

Statistical Analysis

We used descriptive statistics to present data, and Chi-square test and Mann-Whitney U test to compare categorical and numerical variables, respectively, between groups. Because the distributions of NFL levels were skewed, as verified by the Kolmogorov-Smirnov test, we employed the Mann-Whitney U test for the comparisons of NFL levels between the ketamine dependence and control groups or among ketamine-dependent patients with different (low vs. high) levels of NFL after controlling for potential confounders. The comparison of NFL levels between controls and ketamine-dependent patients with and without a lifetime history of MDD were analyzed by ANCOVA with a Tukey-Kramer multiple comparison method. Univariate and multivariate logistic regression analysis was used to examine clinical variables that were associated high NFL levels, which was defined as NFL levels higher than the median values. Bivariate correlation with Spearman's analysis was used to estimate the correlations between NFL levels and the clinical variables including age, BMI, VAS, ketamine use variables, and total scores or number of trauma types of CTQ-SF. A p-value of <0.05 was considered statistically significant. A false discovery rate (FDR) by the Benjamini and Hochberg approach was applied in order to correct for multiple comparisons. Analyses were conducted using SAS statistical software Version 9.4 (SAS Institute, Inc., Cary, N.C.) and GraphPad Prism 5 (GraphPad Software, San Diego, Calif., USA).

### Results

Sample Characteristics

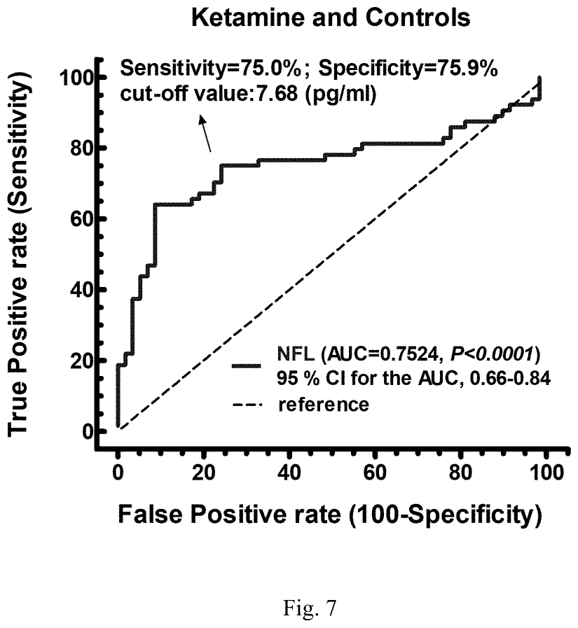

A total of 125 participants, comprising 65 patients with ketamine dependence (56 males and 9 females), and 60 age- and sex-matched controls (51 males and 9 females), were enrolled in our study. Ketamine-dependent patients had significantly lower BMI compared to controls and the percentage of tobacco smokers was significantly higher. All of them reported snorting as the main route of ingestion with none administering by injection. They started using ketamine recreationally, continued using it heavily for an extended period of time, and currently showed high frequency of use during the last month. 56.6% of them reported a lifetime history of MDD. Regarding the comparison of NFL levels between groups, we found, after adjustment for BMI and smoking, that the ketamine dependence group displayed significantly higher NFL levels compared with the controls (14.7±14.6 vs. 7.1±2.8 pg/mL, P<0.001) (see Table 12). FIG. 7 shows a curve graph showing ROC curve analyses of plasma levels of NFL with ketamine patients and controls

Clinical Factors Associated with High NFL Levels

In order to investigate clinical factors that might contribute to NFL elevation, we dichotomized the ketamine dependence group into two groups based on the median level of NFL (10.3 pg/mL) and performed univariate and multi-variate logistic regression analysis. We found age and a lifetime history of MDD were significantly associated with high NFL levels. When we again removed the 8 outliers and used the median level of the remaining ketamine-dependent patients (9.6 pg/mL) as cut-off for dichotomizing the group, we still found age and a lifetime history of MDD as the only significant predictors, with odds ratio 1.20 (95% CI:1.03-1.40, P<0.05) and 9.03 (95% CI:2.14-38.08, P<0.01), respectively.

The difference of NFL levels between ketamine dependence with and without a lifetime history of MDD compared to controls

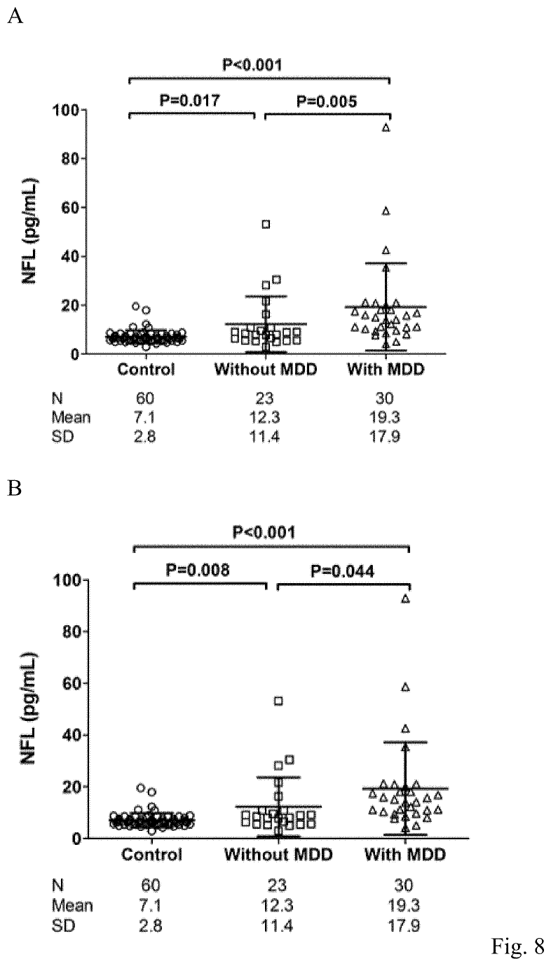

Ketamine-dependent patients with a lifetime history of MDD had a significantly higher NFL levels as confirmed by the Mann-Whitney U test without adjustment (p<0.01) (FIG. 8A), and both subgroups (with and without lifetime MDD) displayed higher NFL levels than control group (P=0.017 and P<0.005 respectively). The Tukey-Kramer multiple comparison test also revealed higher NFL levels in patients with the MDD after adjustment for BMI and smoking status (P<0.05) (FIG. 8B). Clinical variables were similar between MDD and non-MDD subgroups, except that in MDD subgroup the CTQ-SF total scores and number of childhood trauma types were higher (P<0.01 and P<0.05 respectively). In the MDD subgroup, we observed a non-significant trend level increase of NFL levels in those with current MDD symptoms (n=15) compared with those who did not manifest MDD symptoms in the last month (n=38) (23.4±24.0 and 13.4±9.9 pg/mL, respectively; P=0.074) (data not shown), suggesting that NFL might be elevated in patients with currents symptoms of MDD. Thus, the association of NFL levels to current depressive symptoms should further be investigated in larger cohorts of MDD patients.

The Correlation of NFL Levels with Clinical Factors and Ketamine Use Variables

Within the ketamine dependence group, age was the only clinical variable showing significant correlation with NFL levels (P<0.001) (Table 13). A trend level correlation was also observed for the number of childhood trauma types in CTQ-SF (P=0.08) and for ketamine using days in the last 30 days (P=0.07). No significant correlation was observed for all over clinical factors or ketamine use variables, including total years of ketamine use, daily dose or VAS of ketamine craving (p values: all>0.10).

TABLE 12
Sociodemographic and clinical characteristics in treatment-
seeking ketamine-dependent (KD) patients and controls

| | Controls | KD patients | | |
|---|---|---|---|---|
| Variable | (N = 60) | (N = 65) | P value^{a} | FDR^{d} |
| Age, years, mean ± SD | 34.0 ± 5.9 | 32.4 ± 5.9 | 0.13 | 0.16 |
| Sex, N (%) | | | 0.85 | 0.85 |
| Male | 51 (85.0) | 56 (86.2) | | |
| Female | 9 (15.0) | 9 (13.8) | | |
| BMI, kg/m², mean ± SD | 24.7 ± 3.7 | 21.8 ± 3.9 | <0.001 | <0.001 |
| | | (N = 61) | | |
| Current smokers, N (%) | 6 (10.0) | 50 (86.2) | <0.001 | <0.001 |
| Lifetime history of MDD, N (%) | | 30 (56.6) | | |
| | | (N = 53) | | |
| Ketamine use variables, mean ± SD | | | | |
| Total years of ketamine use | | 8.9 ± 4.9 | | |
| | | (N = 58) | | |
| Average daily dose in past 30 days | | 4.5 ± 4.2 | | |
| (g/day) | | (N = 53) | | |
| Maximum daily dose in past 30 days | | 8.9 ± 8.8 | | |
| (g/day) | | (N = 51) | | |
| Using days in past 30 days (days) | | 25.0 ± 10.4 | | |
| | | (N = 50) | | |
| VAS for craving (0-100), mean ± SD | | 27.7 ± 28.8 | | |
| | | (N = 55) | | |
| CTQ-SF | | | | |
| Total scores | | 60.2 ± 15.5 | | |
| | | (N = 48) | | |
| Number of childhood trauma types | | 1.9 ± 1.7 | | |
| | | (N = 48) | | |
| NFL levels (pg/mL) | 7.1 ± 2.8 | 14.7 ± 14.6 | <0.001^{b} | <0.001 |

Abbreviations: BMI: Body Mass Index; CTQ-SF: Childhood Trauma Questionnaire- Short Form; MDD: major depressive disorder; NFL: neurofilament light chain, SD: standard deviation; SDS, Severity of Dependence Scale; VAS, Visual Analogue Scale for craving.
^{a}Mann-Whitney U test used for numerical variables; Chi-square test used for categorical variables.
^{b}Analysis of covariance between KD and control group with BMI and smoking status as covariates.
^{d}FDR: False Discovery Rate for multiple testing.

TABLE 13
The correlation of NFL with clinical parameters and ketamine use variables in the ketamine dependent patients.

| | | | | | Number of | Total Years | Average | | Using |
|---|---|---|---|---|---|---|---|---|---|
| | | | | CTQ-SF | childhood | of | daily dose | Maximum | Days in |
| Bivariate correlation | | | | total | trauma | Ketamine | in past 30 | Dose of | 30 |
| analysis | Age | BMI | VAS | scores | types | Use | days | Ketamine | Days |
| n | 65 | 61 | 55 | 48 | 48 | 58 | 53 | 51 | 50 |
| Spearman's r | 0.484 | −0.198 | 0.088 | 0.190 | 0.252 | 0.083 | −0.020 | 0.092 | 0.259 |
| P value | <0.001 | 0.13 | 0.52 | 0.20 | 0.08 | 0.54 | 0.89 | 0.52 | 0.07 |
| FDR | <0.001 | 0.28 | 0.60 | 0.35 | 0.25 | 0.60 | 0.89 | 0.60 | 0.25 |

Abbreviations: BMI: Body Mass Index; CTQ-SF: Childhood Trauma Questionnaire- Short Form; VAS, Visual Analogue Scale for craving.
FDR: False Discovery Rate for multiple testing.

### Example 7. Peripheral Biomarker Nectin-4 is Increased in Ketamine-Dependent Patients and Correlated with Lower Urinary Tract Symptoms

Participants

The section of Participants is the same as that described in Example 6.

Assay for Serum Nectin-4

A quantitative sandwich enzyme immunoassay technique (R&D Systems, Minneapolis, Minn., USA) was used to detect soluble nectin-4 in the serum of patients with KD and controls. A monoclonal antibody specific for human nectin-4 was precoated onto a microplate, and 50 μL of standards and samples were pipetted into the wells and incubated for 2 h at 2° C.-8° C. After washing away any unbound substances, a cold enzyme-linked polyclonal antibody specific for human nectin-4 was added to the wells for 2 h at 4° C. After washing to remove any unbound antibody-enzyme reagent, a substrate solution was added to the wells for 30 min at room temperature, and a dense color developed in proportion to the amount of nectin-4 bound in the initial step. The stop solution was then added. Wells were read for absorption at 450 nm and 570 nm, and the nectin-4 concentration in the samples was calculated from standard curves. The detection limit for nectin-4 was 16.6 pg/mL.

Statistical Analyses

Descriptive statistics were used to present the data, and the chi-square test and Mann-Whitney U test were used to compare categorical variables and numerical variables, respectively, between the KD and control groups. Bivariate correlation with Spearman's correlation analysis was used to estimate the correlations between nectin-4 levels and the clinical variables including age, BMI, VAS, ketamine use variables, and psychological symptoms. Because the distributions of nectin-4 levels were skewed, as verified by the Shapiro-Wilk normality test, the Mann-Whitney U test was employed to compare nectin-4 levels between the KD and control groups or among patients with KD with different levels of nectin-4 after controlling for potential confounders, including age and BMI. The comparisons of nectin-4 levels between controls and KD patients with and without LUTS were analyzed by ANCOVA with the Tukey-Kramer multiple comparison method. A P value <0.05 was considered statistically significant. Analyses were conducted using SAS version 9.4 (SAS Institute, Inc., Cary, N.C., USA) and GraphPad Prism 5 (GraphPad Software, San Diego, Calif., USA).

### RESULTS

Sample Characteristics

A total of 157 participants (88 patients with KD and 69 gender-matched controls) were enrolled in the study. Their demographic data and clinical characteristics are presented in Table 14. The average age of patients with KD was significantly higher than that of the controls (P=0.004). Gender distribution was equal in the KD and control groups by study design. Patients with KD had a lower BMI (P=0.0001) than controls, and a higher percentage of them were smokers (P<0.0001). All patients with KD had been taking ketamine for an extended period of time (6.61±4.26 years), with relatively high doses (average and maximum daily dose: 3.3±2.32 and 7.01±5.87 g, respectively) and a high frequency of use (26.37±8.38 days) in the 1 month preceding the study. The patients expressed having moderate cravings for ketamine, a high severity of ketamine dependence, and high levels of depression and anxiety. The serum level of nectin-4 was significantly higher in the patients with KD than in controls, as assessed using the immunoassay technique (P<0.0001). Even after adjusting for age, BMI, and smoking, the serum levels of nectin-4 were significantly different between the controls and patients (P<0.0001).

TABLE 14
Sociodemographic and clinical characteristics of patients with KD and normal controls

| | Normal controls | | Patients with KD | | |
|---|---|---|---|---|---|
| | (N = 69) | | (N = 88) | | P-value |
| Age, years, mean ± SD (N) | 28.82 ± 10.57 | | 29.79 ± 6.13 | | 0.004 |
| | (67) | | (86) | | |
| Sex, % (N) | | | | | 0.472 |
| Male | 78.3 | (54) | 73.3 | (63) | |
| Female | 21.7 | (15) | 26.7 | (23) | |
| BMI, Kg/m², mean ± SD (N) | 23.72 ± 3.93 | | 21.23 ± 4.3 | | 0.0001 |
| | (53) | | (79) | | |
| Smokers, N (%) | | | | | <0.0001 |
| No | 41 | (82) | 5 | (6.3) | |
| Yes | 9 | (18) | 74 | (93.7) | |
| Smoking pack-years, mean ± SD (N) | 7.09 ± 5.59 | | 12.77 ± 11.24 | | 0.115 |
| | (9) | | (77) | | |
| Ketamine use parameters, mean ± SD (N) | | | | | |

| | |
|---|---|
| Total years of ketamine use, years | 6.61 ± 4.26 |
| | (81) |
| Average daily dose in past 30 days, g/day | 3.3 ± 2.32 |
| | (72) |
| Maximum daily dose in past 30 days, g/day | 7.01 ± 5.87 |
| | (69) |
| Using days in past 30 days, days | 26.37 ± 8.38 |
| | (73) |
| VAS for craving (0-100), mean ± SD (N) | 33.07 ± 33.88 |
| | (83) |
| SDS score, mean ± SD (N) | 8.98 ± 4.02 |
| | (83) |
| BDI score, mean ± SD (N) | 24.29 ± 12.62 |
| | (84) |
| BAI score, mean ± SD (N) | 15.44 ± 12.96 |
| | (84) |
| OABSS, mean ± SD (N) | 4.84 ± 3.62 |
| | (86) |

| | | | |
|---|---|---|---|
| Serum levels of nectin-4, pg/mL, | 133.98 ± 41.36 | 186.39 ± 57.21 | <0.0001 |
| mean ± SD (N) | (67) | (87) | |

Mann-Whitney U test used for continuous variables; Chi-square test used for categorical variable.
Nectin-4, serum nectin-4 (pg/mL), removed two Controls and one Ketamine outliers.
VAS, Visual Analogue Scale.
SDS, Severity of Dependence Scale.
BDI, Becker Depression Inventory.
Becker Anxiety Inventory.
OABSS, overactive bladder symptoms.

Correlation of Nectin-4 Levels with Clinical Variables in Patients with KD

The correlations between nectin-4 levels and the demographics, ketamine use parameters, and clinical variables of patients with KD are presented in Table 15. The level of nectin-4 was negatively correlated with BMI in patients with KD (P=0.045). Nectin-4 level was not significantly correlated with smoking; ketamine use parameters; VAS for craving; and SDS, BDI, and BAI scores.

TABLE 15
Correlations between nectin-4 levels with clinical
and ketamine use variables in patients with KD

| | | | | P, adjusted for |
|---|---|---|---|---|
| | | | | age, BMI and |
| | n | Nectin-4, r | P | smokers |
| BMI (Kg/m²) | 78 | −0.228 | 0.045 | — |
| Smokers | 78 | 0.080 | 0.485 | — |
| Smoking (pack-years) | 76 | −0.067 | 0.565 | 0.743 |
| Ketamine use parameters | | | | |
| Total years of ketamine use (years) | 80 | −0.060 | 0.599 | 0.133 |
| Average daily dose in past 30 days (g/day) | 71 | 0.023 | 0.848 | 0.852 |
| Maximum daily dose in past 30 days (g/day) | 68 | −0.085 | 0.490 | 0.269 |
| Using days in past 30 days (days) | 72 | −0.020 | 0.870 | 0.507 |
| VAS for craving | 82 | −0.123 | 0.273 | 0.471 |
| SDS | 82 | −0.028 | 0.801 | 0.905 |
| BDI | 83 | 0.011 | 0.922 | 0.881 |
| BAI | 83 | −0.171 | 0.122 | 0.406 |
| OABSS | 85 | −0.103 | 0.348 | 0.881 |

P, Spearman's correlation analysis was used.
P for adjusted were calculated by linear regression analysis.
Nectin-4, serum nectin-4 (pg/mL), removed one outlier in Ketamine.
VAS, Visual Analogue Scale.
SDS, Severity of Dependence Scale.
BDI, Becker Depression Inventory.
Becker Anxiety Inventory.
OABSS, overactive bladder symptoms.

Nectin-4 Levels Among Patients with KD and with and without LUTS

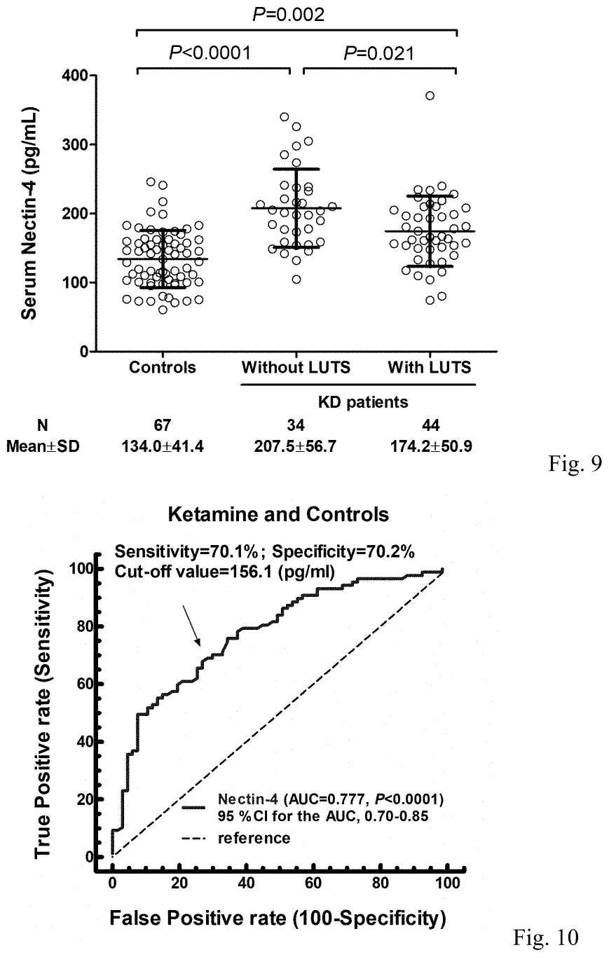

To examine the correlation between nectin-4 level and LUTS in patients with KD, we further divided the patients into two subgroups according to their LUTS. In total, 44 patients had LUTS and 34 patients did not have LUTS. We used the Mann-Whitney U test to determine that the nectin-4 level in patients without LUTS was higher than that in patients with LUTS after adjusting for age, BMI, and smoking (P=0.021; FIG. 9). FIG. 10 shows the ROC curve analyses of serum levels of nectin-4 with ketamine patients and controls.

### Example 8. Peripheral Biomarker CCL11, NFL, MPO is Increased in Alcohol Use Disorder Patients

The investigation was in compliance with the ethical standards described in the declaration of Helsinki and had received approval from the institutional review board at Taipei City Psychiatric Center (TCPC) (IRB No: TCHIRB-10701109) and National Health Research Institutes (Zhunan, Taiwan) (IRB No: EC1070102) before the study began. Patients with alcohol use disorder (AUD) were recruited from an alcohol-detoxification treatment unit in TCPC from September 2018 to February 2020. Patients were invited to participate in the study if they fulfilled the following inclusion criteria: (1) aged being between 20 to 65 years; (2) fulfilling the Diagnostic and Statistical Manual of Mental Disorders, 5th Edition, Text Revision (DSM-5) criteria for alcohol use disorder; (3) having the last alcohol consumption within 24 hours prior to admission.

Healthy Control Subjects

The control group included healthy subjects without known physical and psychiatric illnesses identified in the interview and with normal results for routine laboratory tests for liver function. Those in the control group did not meet the diagnostic criteria for alcohol abuse or dependence in the past and they had not consumed alcohol during the previous three months.

In total, 154 patients with AUD and 117 controls were enrolled.

Plasma CCL11, NFL, and Serum MPO Measurement

The plasma CCL11 were measured using the enzyme-linked immunosorbent assay (ELISA) according to the manufacturer's instruction (R&D Systems Inc, Minneapolis, Minn., USA). Patient's plasma neurofilament light chain (NFL) concentration was measured by quantitative horseradish peroxidase (HRP) Enzyme-linked immunosorbent assay (ELISA) kit (OKCD01380; Aviva Systems Biology, San Diego, Calif., USA). The serum MPO were measured using the enzyme-linked immunosorbent assay (ELISA) according to the manufacturer's instruction (R&D Systems Inc, Minneapolis, Minn., USA).

Result for CCL11, NFL and MPO in Alcohol Dependence (AD) and Control Group

A total of 271 participants (154 patients with AUD and 117 age-gender matched controls) were enrolled in the study. Their demographic data and clinical characteristics are presented in Table 16. The average age of patients with AUD was 45 and controls was 43. Gender distribution was equal in the AUD and control groups by study design. Patients with AUD had a higher pack-years of cigarette smoking (P<0.001) than controls, and higher percentage of smokers (P<0.001). The CCL11 levels were increased in patients with AUD (90.8±32.8 vs. 60.5±16.0 pg/ml, P<0.001; P=0.009 after adjustment for smoking) after removal of 13 outliers. The NFL levels were increased in patients with AUD (231.0±197.1 vs. 72.1±35.6 pg/ml, P<0.001 after adjustment for smoking) removed 12 samples of over detection limit. And the MPO levels were increased in patients with AUD (192.3±113.4 vs. 89.3±56.3 ng/ml, P<0.001 after adjustment for smoking) removed 18 samples of over detection limit.

TABLE 16
Baseline demographic and clinical characteristics
in alcohol dependence (AD) and control group

| | Controls | AUD patients | |
|---|---|---|---|
| Variable | N = 117 | N = 154 | P value |
| Age, mean ± SD | 43.0 ± 10.8 | 45.0 ± 9.1 | 0.08 |
| Gender, N (%) | | | 0.92 |
| Male | 97 (82.9) | 127 (82.5) | |
| Female | 20 (17.1) | 27 (17.5) | |
| Smoking history | | | |
| Ever smokers, N (%) | 12 (10.3) | 130 (85.0) | <0.001 |
| Pack-years of cigarette smoking, mean ± SD | 0.8 ± 3.1 | 23.5 ± 22.6 | <0.001 |
| | (N = 114) | (N = 145) | |
| Alcohol drinking variables, mean ± SD | | | |
| Age of first drink, years old | | 16.9 ± 5.8 | |
| | | (N = 148) | |
| Duration of alcohol dependence (yrs) | | 17.0 ± 10.3 | |
| | | (N = 147) | |
| Average drinking amount in the past one | | 165.4 ± 119.3 | |
| month (gm of pure ethanol/day) | | (N = 111) | |
| Maximum CIWA-Ar scores, mean ± SD | | 15.1 ± 6.1 | |
| | | (N = 84) | |
| Psychological assessment, mean ± SD | | | |
| SADQ | | 23.5 ± 10.7 | |
| | | (N = 84) | |
| VAS (0-100 mm) | | 65.0 ± 31.3 | |
| | | (N = 140) | |
| BDI | | 19.7 ± 12.0 | |
| | | (N = 126) | |
| BAI | | 10.8 ± 8.6 | |
| | | (N = 127) | |
| CCL11 (pg/ml) ^{ζ}, mean ± SD | 60.5 ± 16.0 | 90.8 ± 32.8 | <0.001/0.009^{adj.} |
| | (N = 111) | (N = 145) | |
| NFL (pg/ml) ^{γ}, mean ± SD | 72.1 ± 35.6 | 231.0 ± 197.1 | <0.001/<0.001^{adj.} |
| | (N = 114) | (N = 142) | |
| MPO (ng/ml) ^{ζ}, mean ± SD | 89.3 ± 56.3 | 192.3 ± 113.4 | <0.001/<0.001^{adj.} |
| | (N = 107) | (N = 144) | |

Data are presented as mean ± SD (N). Mann-Whitney U test used for continuous variables; Chi-square test used for categorical variable.
Abbreviations: CIWA-Ar: Clinical Institute Withdrawal Assessment for Alcohol Scale, revised; AST: aspartate transaminase; ALT: alanine transaminase; Bil-T: total bilirubin; γ-GT: gamma-glutamyl transferase; MCV: Mean corpuscular volume; SADQ: Severity of Alcohol Dependence Questionnaire total score; VAS: Visual Analogue Scale; BDI: Beck Depression Inventory; BAI: Beck Anxiety Inventory.
^{ζ} CCL11 (pg/ml) after removal of 13 outliers; MPO (ng/ml) after removal of 18 outliers (i.e. those with a plasma level greater than 1.5 folds of interquartile range)
^{γ} NFL (pg/ml) removed 12 samples of over detection limit,
^{adj.}ANCOVA using pack-years of smoking as the covariate.

### Example 9. The Plasma Biomarkers Associated with Opioid, Ketamine and Alcohol Dependent Patients

Protein Evaluation

The concentration of CCL11 are determined in human blood samples by using the Milliplex MAP human cytokine/chemokine magnetic bead panel kit (Millipore, Billerica, Mass.) or a quantitative sandwich enzyme immunoassay technique (R&D Systems, Minneapolis, Minn.). The analyses are performed according to the protocol of the manufacture. Additionally, a quantitative sandwich enzyme immunoassay technique (R&D Systems, Minneapolis, Minn.) is used to detect soluble nectin-4 in plasma of patients, and the myeloperoxidase (MPO) in serum of patients. On the other hand, patient's plasma neurofilament light chain polypeptide (NFL) concentration is measured by quantitative horseradish peroxidase (HRP) Enzyme-linked immunosorbent assay (ELISA) kit (OKCD01380; Aviva Systems Biology, San Diego, Calif.,) and serum NFL levels are determined at the Cold Spring Biotech Corp. (New Taipei City, Taiwan, R.O.C.) using a SiMoA platform provided by Quanterix (Quanterix Corporation, Billerica, Mass.).

Statistical Analyses

To explore the predicting cut-off value for the plasma level of Nectin-4, CCL11 or NFL or serum level of NFL or MPO in the age- and gender-matched controls and substance use patients, receiver operating characteristic (ROC) analyses were conducted and the area under the curve (AUC) with an associated 95% confidence interval (CI) was calculated. The ROC curves were plotted by GraphPad Prism 5 (GraphPad Software, San Diego, Calif., USA). A P-value less than 0.05 was set as threshold for statistical significance.

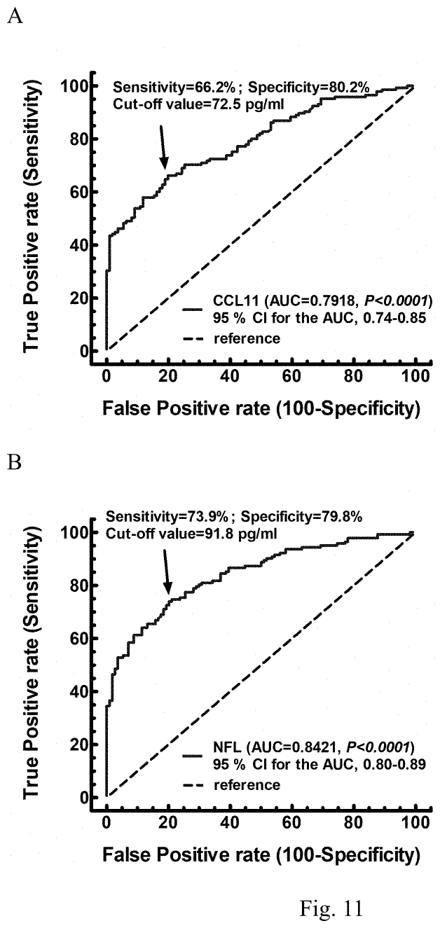

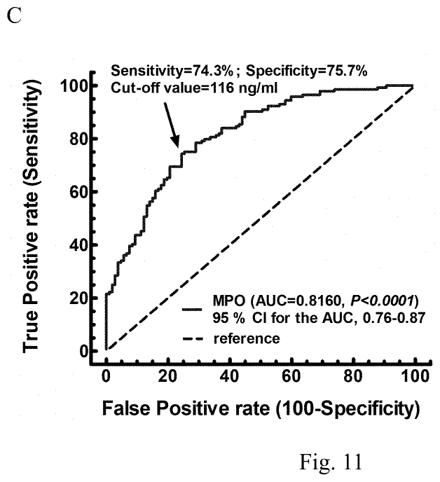

The plasma biomarkers associated with opioid, ketamine and alcohol dependent patients are shown in Table 17. Moreover, FIGS. 11A to 11C show the ROC curve analyses of plasma levels of CCL11, NFL and MPO with alcohol dependence patients and controls.

TABLE 17
Plasma biomarkers associated with opioid, ketamine and alcohol dependent patients

| Plasma | Clinical diagnoses | Substance | ROC curve | | | | |
|---|---|---|---|---|---|---|---|
| molecules | indications | use | Specificity | Sensitivity | AUC | Cut-off value | |
| Nectin-4 | Continual opioid or ketamine | Opioid ^{a} | MMT = 0.73 | MMT = 0.77 | MMT = 0.79 | MMT = 179.6 pg/ml | |
| | use | | skin | skin | skin | skin | |
| | | | irritation = 0.75 | irritation = 0.86 | irritation = 0.86 | irritation = 184 pg/ml | |
| | Severity of dependence | Ketamine ^{a} | 0.70 | 0.70 | 0.78 | 156.1 | pg/ml |
| CCL11 | 1. Neurotoxicity due to | Opioid ^{a} | 0.62 | 0.71 | 0.69 | 73.5 | pg/ml |
| | chronic stress lead to | Alcohol ^{a} | 0.80 | 0.66 | 0.79 | 72.51 | pg/ml |
| | neurodegeneration, | | | | | | |
| | 2. Correlating with severity of | | | | | | |
| | alcohol dependence and | | | | | | |
| | nicotine dependence | | | | | | |
| NFL | 1. Nerve axonal disruption | Alcohol ^{a} | 0.80 | 0.74 | 0.84 | 91.78 | pg/ml |
| | indicating Severity of | Ketamine ^{b} | 0.76 | 0.75 | 0.75 | 7.68 | pg/ml |
| | Neurodegeneration | | | | | | |
| | 2. Correlating with | | | | | | |
| | psychoticism; withdrawal | | | | | | |
| | symptoms, alcohol craving, | | | | | | |
| | obsessive compulsive | | | | | | |
| | drinking, childhood trauma, | | | | | | |
| | highly drinking amount, | | | | | | |
| | depression and anxiety in | | | | | | |
| | alcohol dependents patients | | | | | | |
| | after benzodiazepine | | | | | | |
| | treatment | | | | | | |
| MPO | Alcohol abuse, correlated | Opioid ^{a} | 0.63 | 0.60 | 0.64 | 54.1 | ng/ml |
| | with age of abuse, alcohol | Alcohol ^{a} | 0.76 | 0.74 | 0.82 | 116 | ng/ml |
| | dependence, psychoticism | | | | | | |
| | neutrophil indication | | | | | | |

ROC curve, receiver operating characteristic curve.
AUC, Area under the curve.
^{a} The enzyme-linked immunosorbent assay (ELISA) according to the manufacturer's instruction.
^{b} Single molecule array (SiMoA) immunoassay.

What is claimed is:

1. A method for detecting or predicting a substance use disorder and/or or monitoring a progress of the substance use disorder, and/or predicting treatment response or prognosis of substance use disorder in a subject, comprising obtaining a biological sample, and detecting the level of a biomarker NFL in the biological sample, wherein the level of NFL at least 1.0 times higher than a level of a control is indicative of neurotoxicity severity following substance use, and/or predicting treatment responses or prognosis of substance use disorder in a subject.

2. The method of claim 1, wherein the plasma or serum level of NFL is higher than 5 pg/ml.

3. The method of claim 1, wherein the biomarker further comprises one or more additional biomarkers selected from CCL11 for chronic stress following substance use, Nectin-4 for skin irritation following substance use, MPO for alcohol dependence following substance use and ADAM10 for synaptopathy following substance use.

4. The method of claim 3, wherein the biomarker comprises a combination of NFL and CCL11; NFL, CCL11 and Nectin-4; NFL, CCL11, Nectin-4 and MPO; or NFL, CCL11, Nectin-4, MPO and ADAM10.

5. The method of claim 4, wherein in a subject with a substance use disorder, chronic stress is indicated when the level of CCL11 in a biological sample is at least 1 time higher than that in a control; skin irritation is indicated when the level of Nectin-4 in a biological sample is at least 1 time higher than that in a control; alcohol dependence is indicated when the level of MPO in a biological sample is at least 1 time higher than that in a control; or synaptopathy is indicated when the level of ADAM10 in a biological sample is at least 1 time higher than that in a control

6. The method of claim 4, wherein the plasma or serum level of CCL11 is higher than 40 pg/ml
and/or the plasma or serum level of Nectin-4 is higher than 50 pg/ml,
and/or the plasma or serum level of MPO is higher than 30 pg/ml,
and/or the plasma or serum level of ADAM10 is higher than 15 ng/ml.

7. (canceled)

8. (canceled)

9. (canceled)

10. The method of claim 1, wherein the substance use disorder is drug addiction, drug abuse, drug habituation, drug dependence, withdrawal syndrome or overdose.

11. The method of claim 1, wherein the substance is alcohol, ketamine, opiate, opioid, cocaine, morphine, amphetamines, nicotine, cotinine, heroin, amphetamine, methamphetamine, cannabis, cannabinoid, narcotic analgesic combinations, drug overdose, or chronic substance use.

12. The method of claim 1, wherein the level of the biomarker in a biological sample is detected by incubating a protein or a peptide of the biomarker in the sample with an antibody specifically binding the protein or the peptide and measuring the level of the protein or the peptide.

13. The method of claim 1, wherein the level is measured by immunofluorescent assay, enzyme immunoassay or radioimmunoassay.

14. The method of claim 1, wherein the biological sample is plasma, a tissue, cell, blood, urine or serum.

15. The method of claim 14, wherein the biological sample is plasma or serum.

16. The method of claim 1, wherein the subject has neurotoxicity.

17. The method of claim 1, wherein the subject suffers from infection caused by hepatitis virus or human immunodeficiency virus.

18. A kit for detecting or predicting a substance use disorder and/or or monitoring a progress of the substance use disorder, and/or predicting treatment response or prognosis of substance use disorder, comprising an antibody specifically binding the protein or the peptide of the biomarkers of claim 1 and a second antibody specifically binding the protein or the peptide.

19. The kit of claim 18, which is for detecting or predicting a combined infectious disease following substance use disorder.

20. A method for detecting or predicting an infection caused by hepatitis virus or human immunodeficiency virus and/or or monitoring a progress of the infection, and/or predicting treatment response or prognosis of the infection in a subject, comprising obtaining a biological sample, and detecting the level of a biomarker IP-10 and optional CDH2, IL-7 and/or Caspase-10 or any combination thereof in the biological sample, wherein an level of the biomarker higher than a level of a control is indicative of the infection, or treatment response, progress or prognosis of the infection.

21. The method of claim 20, wherein the hepatitis virus is hepatitis B virus (HBV) or hepatitis C virus (HCV).

22. The method of claim 20, wherein the biomarker comprises IP-10 and CDH2; IP-10, CDH2 and IL-7; or IP-10, CDH2, IL-7 and Caspase-10; IP-10, CDH2 and IL-7; IP-10, CDH2 and Caspase-10; or IP-10, IL-7 and Caspase-10.

23. A kit for detecting or predicting an infection caused by hepatitis virus or human immunodeficiency virus and/or or monitoring a progress of the infection, and/or predicting treatment response or prognosis of the infection, comprising an antibody specifically binding the protein or the peptide of the biomarkers of claim 17 and a second antibody specifically binding the protein or the peptide.
